(19) United States
(12) Patent Application Publication
(10) Pub. No.: US 2021/0393628 A1
(43) Pub. Date: Dec. 23, 2021

(54) COMPOSITIONS AND METHODS FOR MODULATING T CELL EXHAUSTION

(71) Applicant: The Board of Trustees of the Leland Stanford Junior University, Stanford, CA (US)

(72) Inventors: Crystal Mackall, Stanford, CA (US); Vineet Kumar, Stanford, CA (US); Mallesh Pandrala, Stanford, CA (US); Evan Weber, Stanford, CA (US); Sanjay Malhotra, Stanford, CA (US)

(21) Appl. No.: 17/290,111

(22) PCT Filed: Oct. 30, 2019
(86) PCT No.: PCT/US2019/058966

§ 371 Date: Apr. 29, 2021

Related U.S. Application Data
(60) Provisional application No. 62/752,401, filed on Oct. 30, 2018.

(51) Int. Cl. A61K 31/506; A61K 35/17; A61K 31/5025; A61K 45/06; C07D 417/14; A61P 35/00

(52) U.S. Cl. CPC A61K 31/506; A61K 35/17; A61P 35/00; A61K 45/06; C07D 417/14; A61K 31/5025

(57) ABSTRACT

This invention is in the field of medicinal chemistry. In particular, provided herein are compositions and methods for preventing or reversing T cell exhaustion. In certain embodiments, the present invention relates to methods of preventing or reversing T cell exhaustion by exposing T cells experiencing T cell exhaustion to a new class of small-molecules having a thiazole, imidazolepyridiazine or piperazinyl-methyl-aniline structure, or by expanding genetically engineered T cells in the presence of such small molecules.

### FIELD OF THE INVENTION

This invention is in the field of medicinal chemistry. In particular, provided herein are compositions and methods for preventing or reversing T cell exhaustion. In certain embodiments, the present invention relates to methods of preventing or reversing T cell exhaustion by exposing T cells experiencing T cell exhaustion to a new class of small-molecules having a thiazole, imidazolepyridiazine or piperazinyl-methyl-aniline structure, or by expanding genetically engineered T cells in the presence of such small molecules.

### INTRODUCTION

T cells are immune cells that become activated via T cell receptor (TCR) signaling following engagement with antigen. Physiologic activation through the T cell receptor renders T cells capable of mediating potent antitumor or anti-infective effects. During resolution of an acute inflammatory response, a subset of activated effector T cells differentiate into long-lived memory cells. By contrast, in patients with chronic infections or cancer, T cells not infrequently undergo pathologic differentiation toward a state of dysfunction, which has been termed T cell exhaustion. T cell exhaustion is characterized by marked changes in metabolic function, transcriptional programming, loss of effector function (e.g., cytokine secretion, killing capacity), and co-expression of multiple surface inhibitory receptors. The root cause of T cell exhaustion is persistent antigen exposure leading to continuous TCR signaling. Prevention or reversal of T cell exhaustion has been long sought as a means to enhance T cell effectiveness in patients with cancer or chronic infections.

The present invention addresses this urgent need.

### SUMMARY OF THE INVENTION

Immune cells respond to the presence of foreign antigens with a wide range of responses, including the secretion of preformed and newly formed mediators, phagocytosis of particles, endocytosis, cytotoxicity against target cells, as well as cell proliferation and/or differentiation. T cells are a subgroup of cells which together with other immune cell types (e.g., polymorphonuclear, eosinophils, basophils, mast cells, B cells, and NK cells), constitute the cellular component of the immune system (see, e.g., U.S. Pat. No. 6,057,294; US Pat. Appl. 20050070478). Under physiological conditions T cells function in immune surveillance and in the elimination of foreign antigen. However, under pathological conditions there is compelling evidence that T cells play a major role in the causation and propagation of disease. In these disorders, breakdown of T cell immunological tolerance, either central or peripheral is a fundamental process in the causation of autoimmune disease.

It is well established that T cell receptor (TCR) engagement and costimulatory signaling provide the critical signals that regulate T cell activation, proliferation and cytolytic functions. T cells respond to antigen via a polypeptide complex composed of the ligand-binding T cell receptor (TCR) disulfide-linked α and β subunits (or γ and δ subunits in γδ T cells) that have single transmembrane (TM) spans per subunit and small intracellular tails and associate non-covalently with hetero- (CD3γε and CD3δε) and homodimeric (ζζ) signaling subunits (see, e.g., Cambier J. C. Curr Opin Immunol 1992; 4:257-64). The CD3ε, δ, and γ chains have single Ig-family extracellular domains, single presumably α-helical TM spans, and intrinsically disordered intracellular domains of 40-60 residues, whereas each ζ subunit has a small extracellular region (9 residues) carrying the intersubunit disulfide bond, a single presumably α-helical TM span per subunit, and a large, intrinsically disordered cytoplasmic domain of approximately 110 residues. An understanding of the process of TCR-mediated TM signal transduction and subsequent T cell activation, leading to T cell proliferation and differentiation, is therefore pivotal to both health and disease. Disturbance in TCR signaling can lead to inflammatory and other T cell-related disorders.

T cells expressing chimeric antigen receptors (CARs) at high levels undergo tonic, antigen independent signaling due to receptor clustering. Such T cells function poorly as a result of T cell exhaustion, as evidenced by high levels of PD-1, TIM-3, LAG-3, diminished antigen induced cytokine production, and excessive programmed cell death. Tonic signaling can be prevented by transiently decreasing CAR associated TCR signaling proteins (e.g., TCR zeta) to levels below the threshold required for tonic signaling.

It has been shown that treatment with a particular tyrosine kinase inhibitor that inhibits T cell receptor signaling (e.g., a Lck tyrosine kinase inhibitor (e.g., dasatinib)) (e.g., a Src family tyrosine kinase inhibitor) reduced expression of the T cell exhaustion markers and improved formation of T cell memory (see, e.g., International Patent Application Publication No. 2018/183842). It has been shown that CAR T cells co-cultured with tumor cells in the presence of dasatinib or ponatinib exhibit attenuated activation and degranulation, fail to secrete cytokine, and display attenuated killing in response to tumor antigen (see, e.g., International Patent Application Publication No. 2018/183842). It has been shown that dasatinib potently inhibits the phosphorylation of CAR CD3z as well as distal signaling proteins after CAR crosslinking (see, e.g., International Patent Application Publication No. 2018/183842). It has been shown that tonically signaling CAR T cells expanded in the presence of dasatinib exhibit a reduction in canonical exhaustion marker expression in a dose-dependent manner, retain the capacity to form memory, display augmented cytokine secretion in response to tumor antigen, and display augmented cytotoxicity (see, e.g., International Patent Application Publication No. 2018/183842). It has been shown that in vivo dasatinib treatment suppresses exhaustion marker expression, augments memory formation, and facilitates cell survival/proliferation (see, e.g., International Patent Application Publication No. 2018/183842).

As indicated, experiments conducted during the course of developing embodiments for the present invention synthesized certain thiazole, imidazolepyridiazine and piperazinyl-methyl-aniline compounds and determined that such compounds function as modulators of CAR-T cell activity and effects related to CAR-T cell activity (e.g., preventing or reversing T cell exhaustion), and serve as therapeutics for use in CAR-T cell based therapies. For example, such experiments determined exposure of healthy donor purified T cells that were artificially conditioned to become exhausted ex vivo by transducing them to express a CAR that tonically signals in the absence of antigen with either a compound of the present invention or a tyrosine kinase inhibitor resulted in increased CAR-T cell expression of POLDIP2, GSTK1, and STMN2, and decreased CAR-T cell expression of GZMB, MAPRE2, NAMPT, and SIGMAR1. Moreover, additional experiments were conducted to assess the effects of the compounds recited herein on CAR T cell antigen-induced activation. Of the 27 compounds tested, 13 induced measurable suppression of CD69 and CD107a at the highest tested concentration of 10 uM, and 9 (EB1P083, EB1P084, EB1P085, EB1P086, EB1P088, EB1P089, EB1P090, EB1P091, EB2P067) induced measurable suppression at 1 uM. EB1P084, EB1P085, EB1P088, EB1P089, and EB2P067 exhibited the greatest potency at the 1 uM concentration compared to others.

Thus, the present invention relates to methods of preventing or reversing T cell exhaustion by exposing T cells experiencing T cell exhaustion to a new class of small-molecules having a thiazole, imidazolepyridiazine or piperazinyl-methyl-aniline structure, or by expanding genetically engineered T cells in the presence of such small molecules.

Certain thiazole, imidazolepyridiazine and piperazinyl-methyl-aniline compounds of the present invention may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

In a particular embodiment, thiazole compounds having Formula I, imidazolepyridiazine compounds having Formula II, and piperazinyl-methyl-aniline compounds having Formula III are provided as modulators of CAR-T cell activity and effects related to CAR-T cell activity (e.g., preventing or reversing T cell exhaustion):

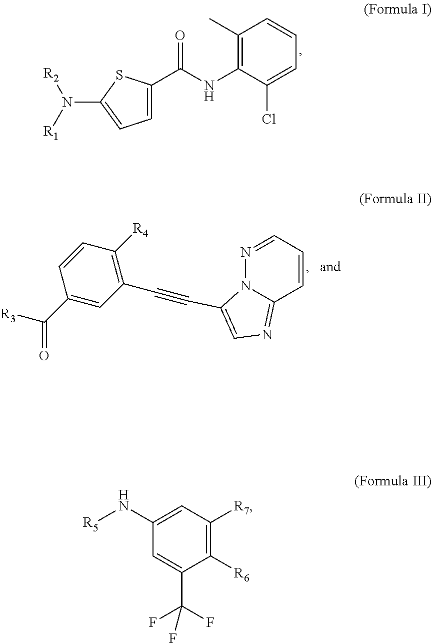

including pharmaceutically acceptable salts, solvates, and/or prodrugs thereof.

Formulas I, II and III are not limited to a particular chemical moiety for R1, R2, R3, R4, R5, R6 and R7. In some embodiments, the particular chemical moiety for R1, R2, R3, R4, R5, R6 and R7 independently include any chemical moiety that permits the resulting compound to increase CAR-T cell expression of one or more of POLDIP2, GSTK1, and STMN2. In some embodiments, the particular chemical moiety for R1, R2, R3, R4, R5, R6 and R7 independently include any chemical moiety that permits the resulting compound to decrease CAR-T cell expression of one or more of GZMB, MAPRE2, NAMPT, and SIGMAR1. In some embodiments, the particular chemical moiety for R1, R2, R3, R4, R5, R6 and R7 independently include any chemical moiety that permits the resulting compound to modulate (e.g., inhibit) CAR-T cell activity. In some embodiments, the particular chemical moiety for R1, R2, R3, R4, R5, R6 and R7 independently include any chemical moiety that permits the resulting compound to modulate (e.g., inhibit) TCR or CAR-mediated signaling related to antigen-dependent or antigen-independent CAR T cell activation. In some embodiments, the particular chemical moiety for R1, R2, R3, R4, R5, R6 and R7 independently include any chemical moiety that permits the resulting compound to prevent and/or reverse T cell exhaustion related to antigen-dependent or antigen-independent CAR T cell activation. In some embodiments, the particular chemical moiety for R1, R2, R3, R4, R5, R6 and R7 independently include any chemical moiety that permits the resulting compound to decrease CAR-T cell expression of one or more of PD-1, TIM-3, and LAG-3. In some embodiments, the particular chemical moiety for R1, R2, R3, R4, R5, R6 and R7 independently include any chemical moiety that permits the resulting compound to increase CAR-T cell expression of memory markers (e.g., CD62L). In some embodiments, the particular chemical moiety for R1, R2, R3, R4, R5, R6 and R7 independently include any chemical moiety that permits the resulting compound to prevent CAR-T cell apoptosis. In some embodiments, the particular chemical moiety for R1, R2, R3, R4, R5, R6 and R7 independently include any chemical moiety that permits the resulting compound to decrease CAR-T cell secretion of IL-2 and other cytokines. In some embodiments, the particular chemical moiety for R1, R2, R3, R4, R5, R6 and R7 independently include any chemical moiety that permits the resulting compound to decrease CAR-T cell secretion of IL-2 and other cytokines. In some embodiments, the particular chemical moiety for R1, R2, R3, R4, R5, R6 and R7 independently include any chemical moiety that permits the resulting compound to increase CAR-T cell secretion of IL-2 and other cytokines following transient treatment with such a compound and subsequent clearance of compound.

In some embodiments, R1 and R2 are independently selected from hydrogen,

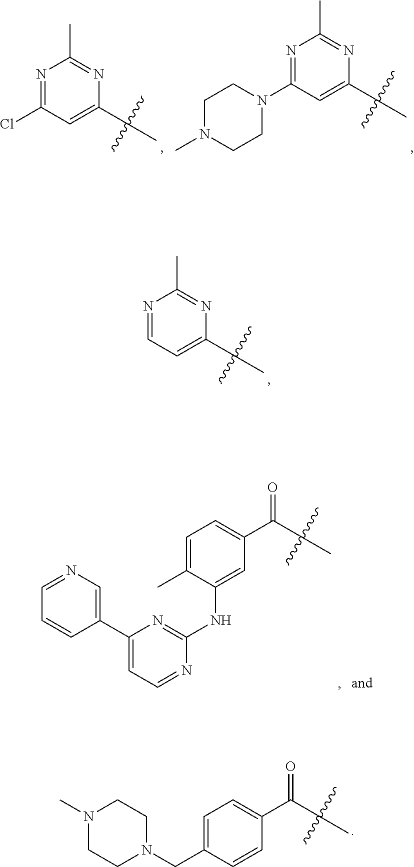

In some embodiments, R3 is selected from hydrogen, hydroxyl,

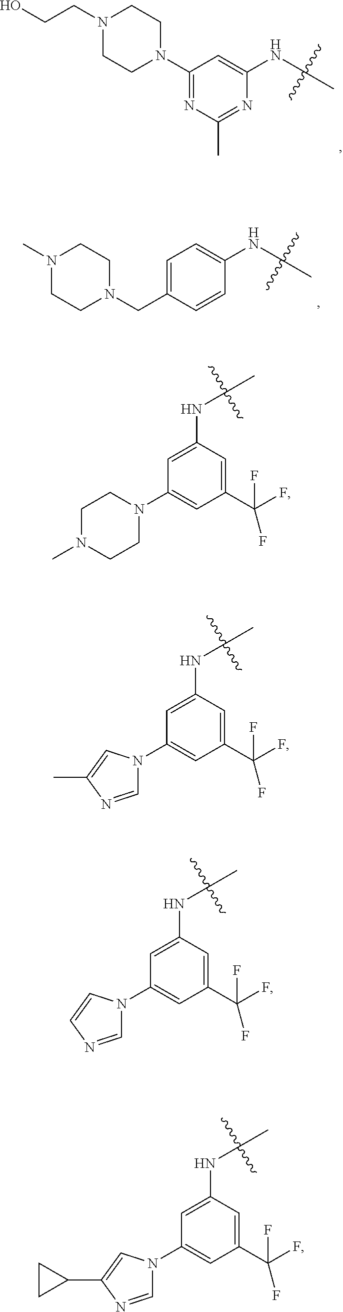 [SMILES: *Nc1cc(N2CCN(CCO)CC2)nc(C)n1 ; *Nc1ccc(CN2CCN(C)CC2)cc1 ; *Nc1cc(N2CCN(C)CC2)cc(C(F)(F)F)c1 ; *Nc1cc(-n2cnc(C)c2)cc(C(F)(F)F)c1 ; *Nc1cc(-n2ccnc2)cc(C(F)(F)F)c1 ; *Nc1cc(-n2cnc(C3CC3)c2)cc(C(F)(F)F)c1]

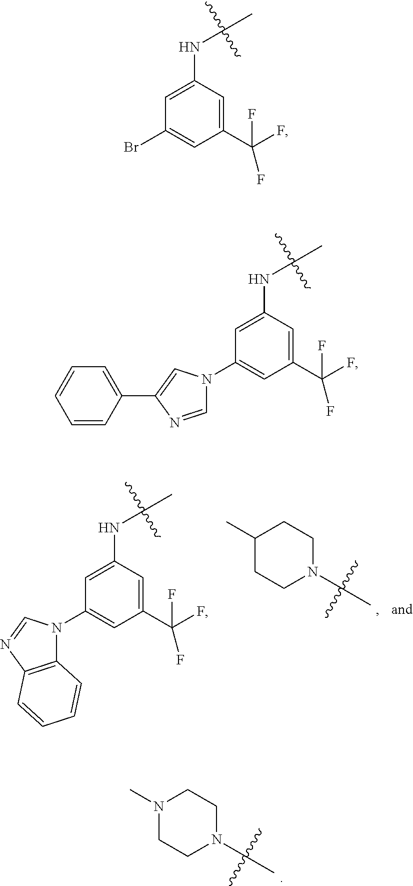

In some embodiments, R4 is hydrogen, methyl or

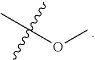 [SMILES: *OC]

In some embodiments, R5 is selected from hydrogen,

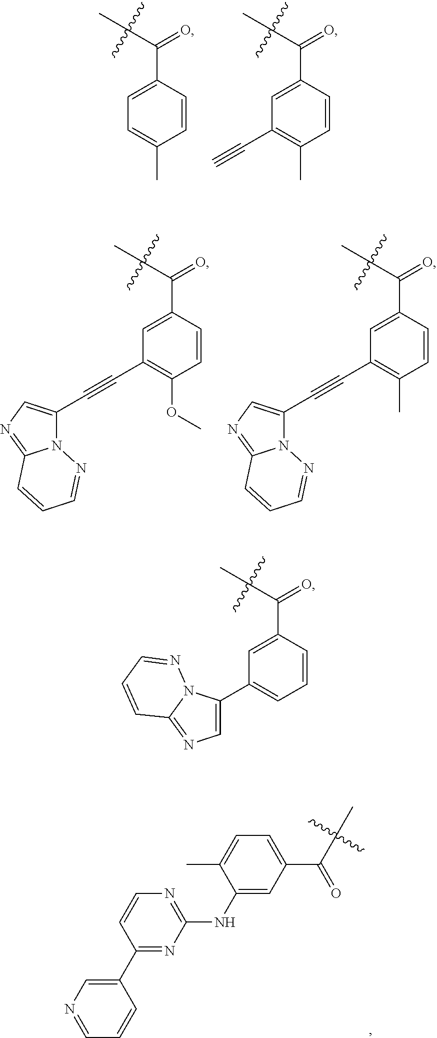

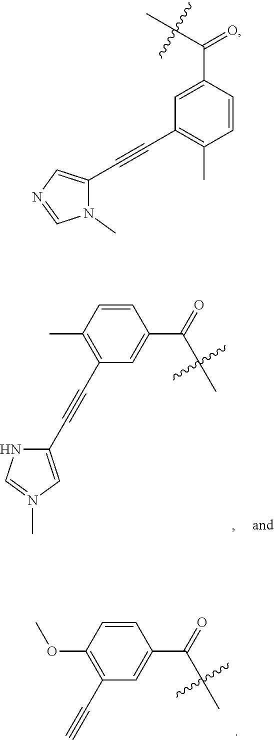

In some embodiments, R6 is hydrogen or

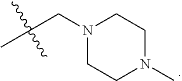 [SMILES: *CN1CCN(C)CC1]

In some embodiments, R7 is hydrogen or

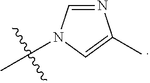 [SMILES: *n1cnc(C)c1]

In some embodiments, the following thiazole, imidazolepyridiazine and piperazinyl-methyl-aniline compounds are contemplated for Formulas I, II and III:

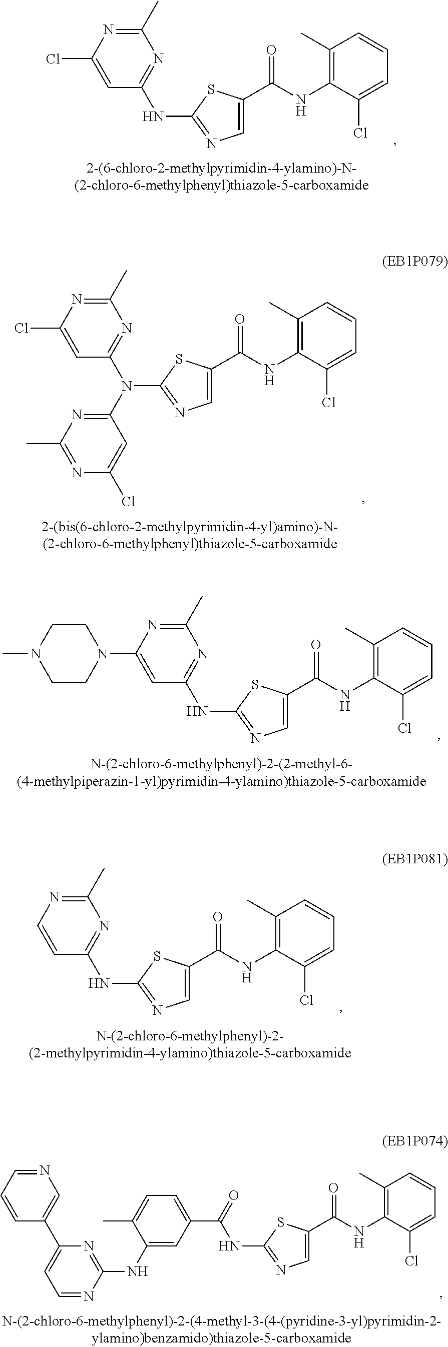 [SMILES: Cc1nc(Cl)cc(Nc2ncc(C(=O)Nc3c(C)cccc3Cl)s2)n1 ; Cc1nc(Cl)cc(N(c2cc(Cl)nc(C)n2)c2ncc(C(=O)Nc3c(C)cccc3Cl)s2)n1 ; Cc1nc(Nc2ncc(C(=O)Nc3c(C)cccc3Cl)s2)cc(N2CCN(C)CC2)n1 ; Cc1nccc(Nc2ncc(C(=O)Nc3c(C)cccc3Cl)s2)n1 ; Cc1ccc(C(=O)Nc2ncc(C(=O)Nc3c(C)cccc3Cl)s2)cc1Nc1nccc(-c2cccnc2)n1]

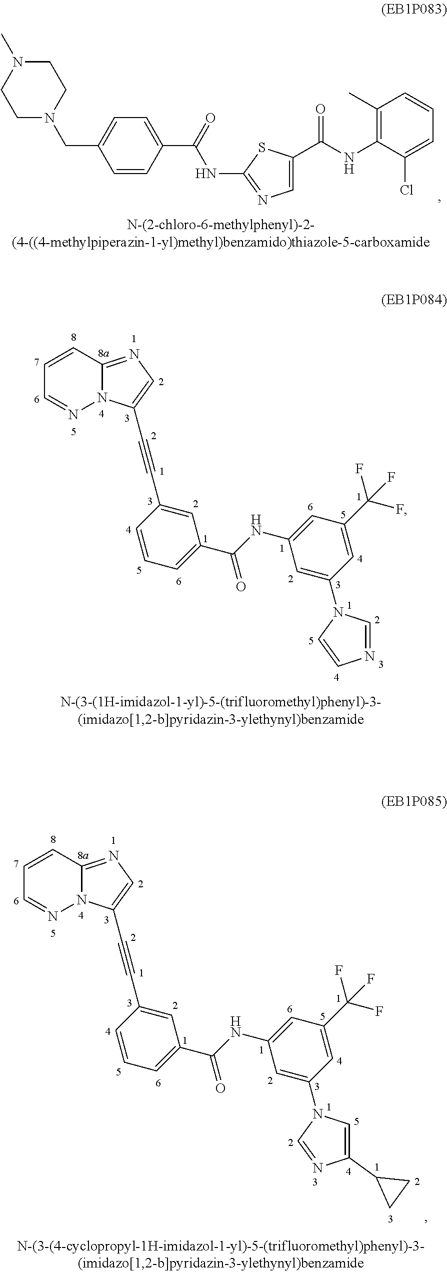 [SMILES: Cc1cccc(Cl)c1NC(=O)c1cnc(NC(=O)c2ccc(CN3CCN(C)CC3)cc2)s1 ; O=C(Nc1cc(-n2ccnc2)cc(C(F)(F)F)c1)c1cccc(C#Cc2cnc3cccnn23)c1 ; O=C(Nc1cc(-n2cnc(C3CC3)c2)cc(C(F)(F)F)c1)c1cccc(C#Cc2cnc3cccnn23)c1]

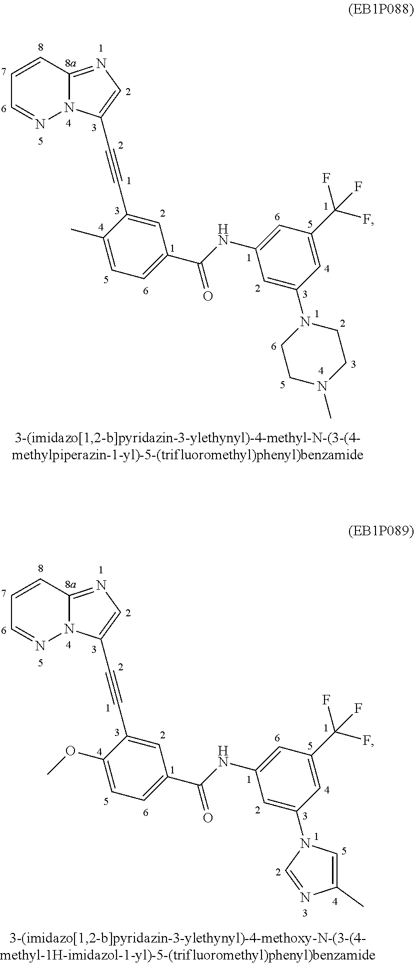 [SMILES: Cc1ccc(C(=O)Nc2cc(N3CCN(C)CC3)cc(C(F)(F)F)c2)cc1C#Cc1cnc2cccnn12 ; COc1ccc(C(=O)Nc2cc(-n3cnc(C)c3)cc(C(F)(F)F)c2)cc1C#Cc1cnc2cccnn12]

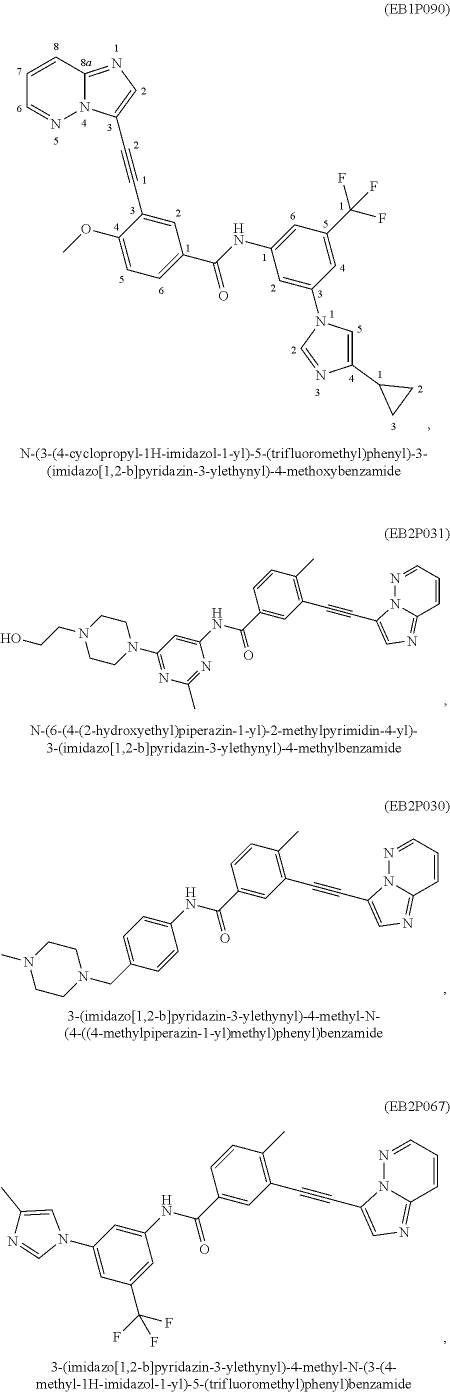 [SMILES: COc1ccc(C(=O)Nc2cc(-n3cnc(C4CC4)c3)cc(C(F)(F)F)c2)cc1C#Cc1cnc2cccnn12 ; Cc1nc(NC(=O)c2ccc(C)c(C#Cc3cnc4cccnn34)c2)cc(N2CCN(CCO)CC2)n1 ; Cc1ccc(C(=O)Nc2ccc(CN3CCN(C)CC3)cc2)cc1C#Cc1cnc2cccnn12 ; Cc1cn(-c2cc(NC(=O)c3ccc(C)c(C#Cc4cnc5cccnn45)c3)cc(C(F)(F)F)c2)cn1]

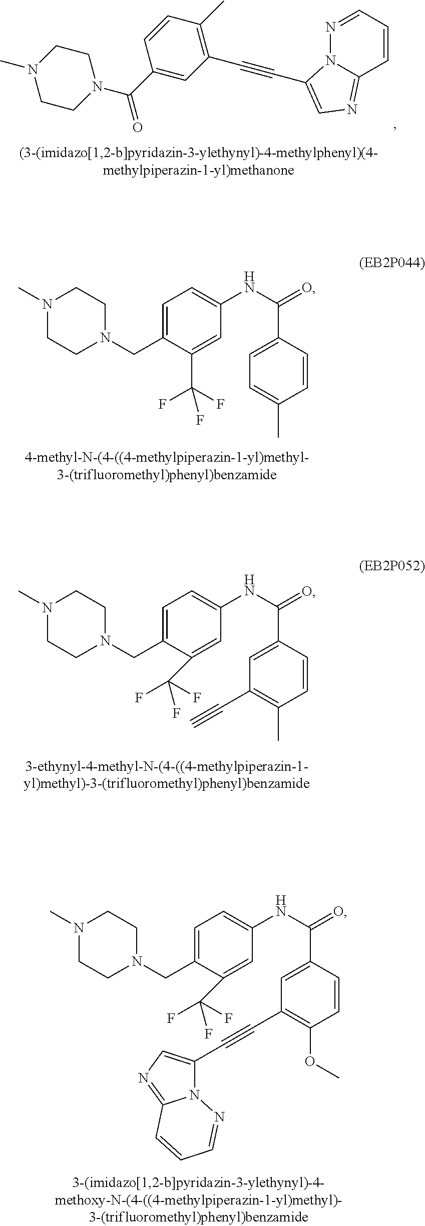

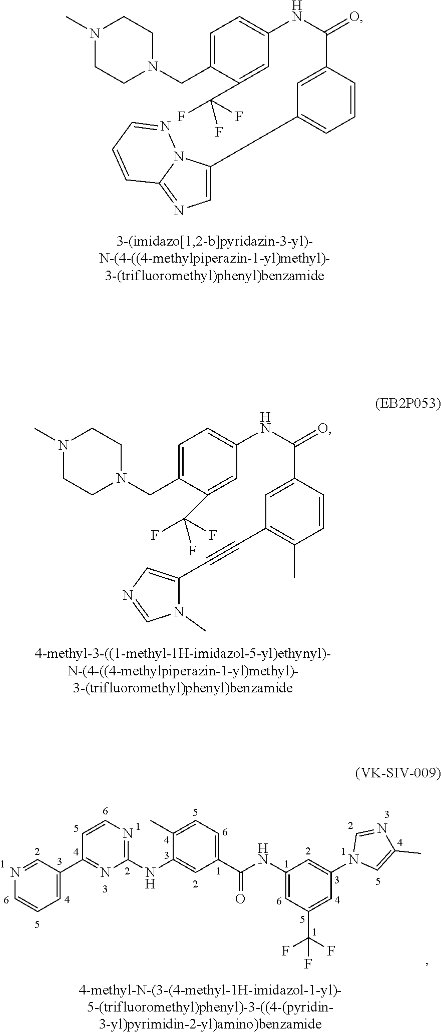 [SMILES: CN1CCN(Cc2ccc(NC(=O)c3cccc(CCc4cnc5cccnn45)c3)cc2C(F)(F)F)CC1 ; Cc1ccc(C(=O)Nc2ccc(CN3CCN(C)CC3)c(C(F)(F)F)c2)cc1C#Cc1cncn1C ; Cc1cn(-c2cc(NC(=O)c3ccc(C)c(Nc4nccc(-c5cccnc5)n4)c3)cc(C(F)(F)F)c2)cn1]

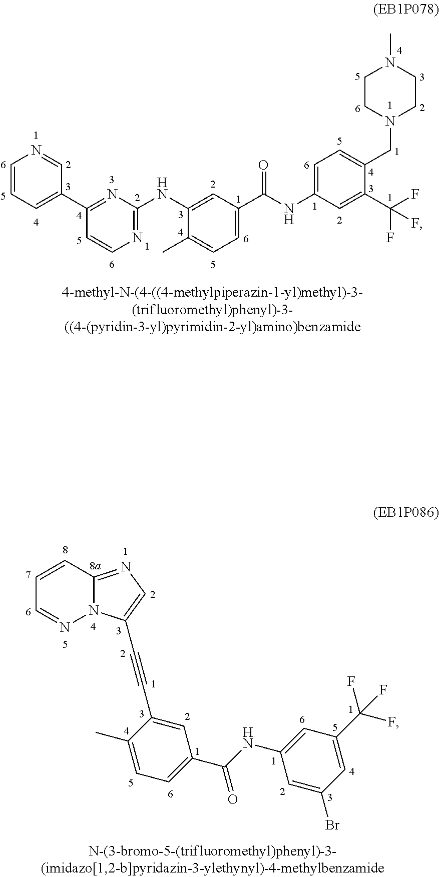 [SMILES: Cc1ccc(C(=O)Nc2ccc(CN3CCN(C)CC3)c(C(F)(F)F)c2)cc1Nc1nccc(-c2cccnc2)n1 ; Cc1ccc(C(=O)Nc2cc(Br)cc(C(F)(F)F)c2)cc1C#Cc1cnc2cccnn12]

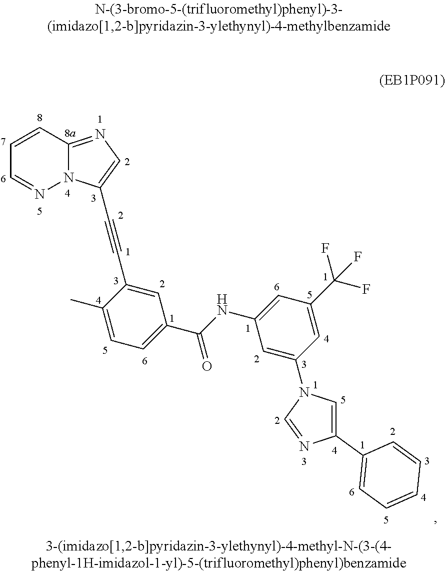 [SMILES: Cc1ccc(C(=O)Nc2cc(-n3cnc(-c4ccccc4)c3)cc(C(F)(F)F)c2)cc1C#Cc1cnc2cccnn12]

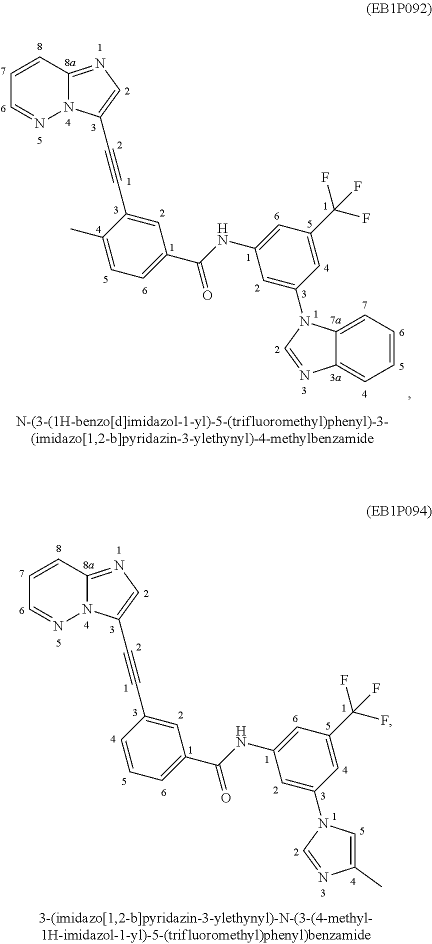 [SMILES: Cc1ccc(C(=O)Nc2cc(-n3cnc4ccccc43)cc(C(F)(F)F)c2)cc1C#Cc1cnc2cccnn12 ; Cc1cn(-c2cc(NC(=O)c3cccc(C#Cc4cnc5cccnn45)c3)cc(C(F)(F)F)c2)cn1]

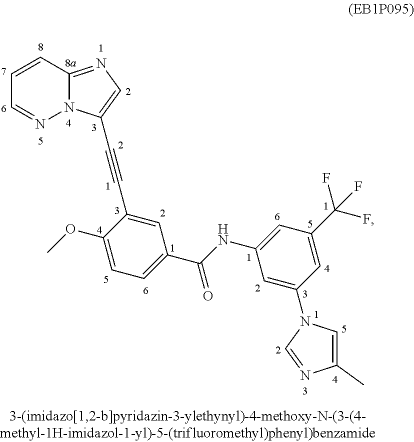 [SMILES: COc1ccc(C(=O)Nc2cc(-n3cnc(C)c3)cc(C(F)(F)F)c2)cc1C#Cc1cnc2cccnn12]

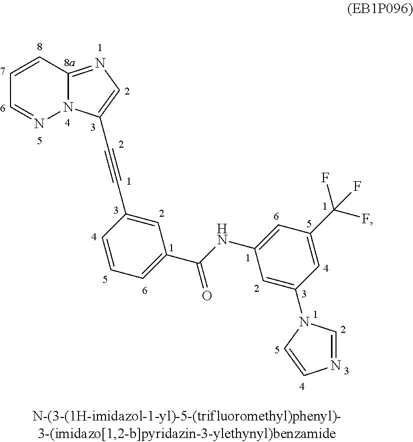 [SMILES: O=C(Nc1cc(-n2ccnc2)cc(C(F)(F)F)c1)c1cccc(C#Cc2cnc3cccnn23)c1]

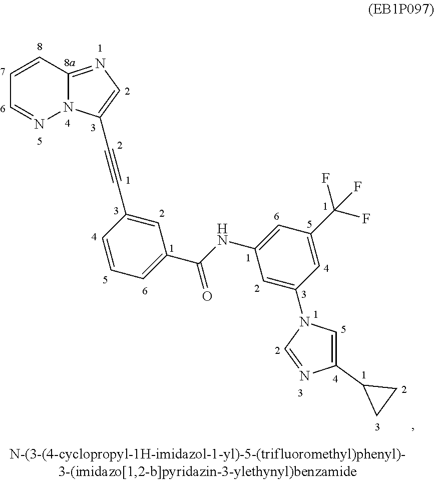 [SMILES: O=C(Nc1cc(-n2cnc(C3CC3)c2)cc(C(F)(F)F)c1)c1cccc(C#Cc2cnc3cccnn23)c1]

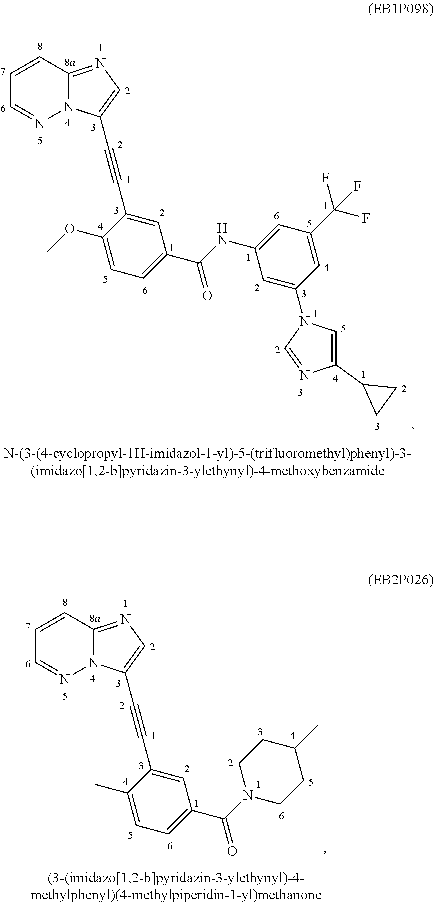 [SMILES: COc1ccc(C(=O)Nc2cc(-n3cnc(C4CC4)c3)cc(C(F)(F)F)c2)cc1C#Cc1cnc2cccnn12 ; Cc1ccc(C(=O)N2CCC(C)CC2)cc1C#Cc1cnc2cccnn12]

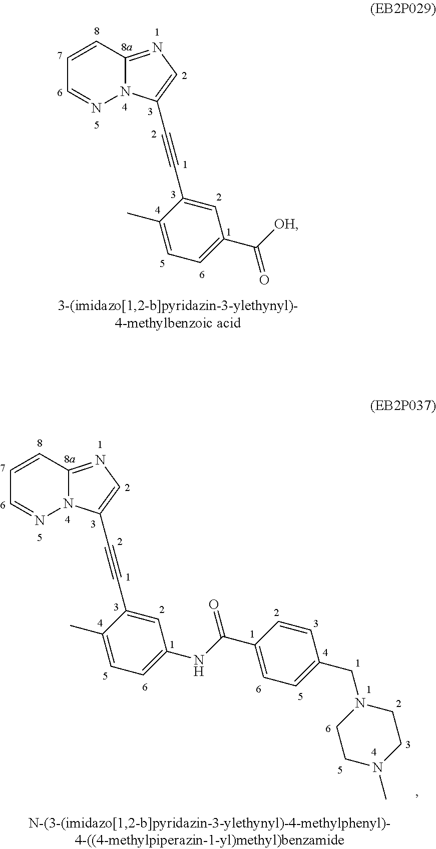 [SMILES: Cc1ccc(C(=O)O)cc1C#Cc1cnc2cccnn12 ; Cc1ccc(NC(=O)c2ccc(CN3CCN(C)CC3)cc2)cc1C#Cc1cnc2cccnn12]

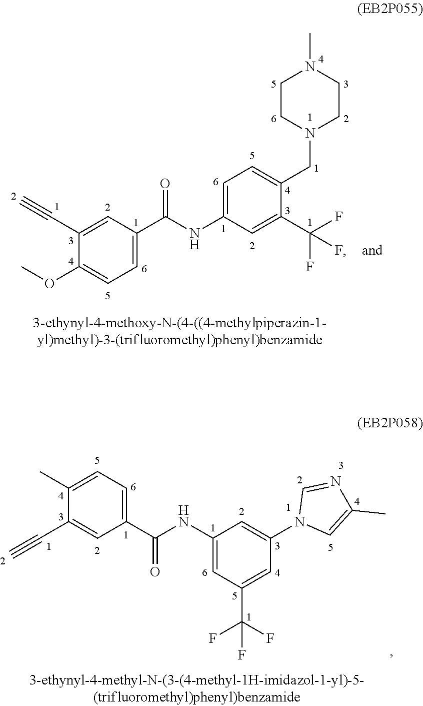 [SMILES: C#Cc1cc(C(=O)Nc2ccc(CN3CCN(C)CC3)c(C(F)(F)F)c2)ccc1OC ; C#Cc1cc(C(=O)Nc2cc(-n3cnc(C)c3)cc(C(F)(F)F)c2)ccc1C]

and including pharmaceutically acceptable salts, solvates, and/or prodrugs thereof.

The invention further provides processes for preparing any of the compounds of the present invention through following any technique known to those of skill in a related art.

In certain embodiments, the present invention provides a pharmaceutical composition comprising a compound of the present invention (e.g., a compound having a thiazole, imidazolepyridiazine or piperazinyl-methyl-aniline structure).

In certain embodiments, the present invention provides methods for treating a subject to mitigate T cell exhaustion, the method comprising administering to the subject a therapeutically effective amount of a pharmaceutical composition comprising a compound of the present invention (e.g., a compound having a thiazole, imidazolepyridiazine or piperazinyl-methyl-aniline structure).

Such methods are not limited to a particular manner of treating the subject for T cell exhaustion. In some embodiments, the treatment increases CAR-T cell expression of one or more of POLDIP2, GSTK1, and STMN2. In some embodiments, the treatment decreases CAR-T cell expression of one or more GZMB, MAPRE2, NAMPT, and SIGMAR1. In some embodiments, the treatment decreases secretion of IL-2 by T cells in the subject. In some embodiments, the treatment decreases apoptosis of T cells in the subject. In some embodiments, the treatment decreases expression of at least one T cell exhaustion marker selected from the group consisting of PD-1, TIM-3, and LAG-3. In some embodiments, the treatment increases expression of CD62L or CCR7. In some embodiments, the treatment decreases T cell secretion of IL-2 and other cytokines. In some embodiments, the treatment increases T cell secretion of IL-2 and other cytokines following transient treatment with such a pharmaceutical composition and subsequent clearance of the pharmaceutical composition.

Such methods are not limited to particular manner of administration. In some embodiments, multiple cycles of treatment are administered to the subject. In some embodiments, the pharmaceutical composition is administered intermittently. In some embodiments, the pharmaceutical composition is administered for a period of time sufficient to restore at least partial T cell function then discontinued. In some embodiments, the pharmaceutical composition is administered orally.

In some embodiments, such pharmaceutical compositions are administered iteratively for purposes of facilitating periods of CAR-T cell inactivation (e.g., during pharmaceutical composition administration) and periods of CAR-T cell activation (e.g., during absence of pharmaceutical composition administration; following clearance of the pharmaceutical composition).

Such methods are not limited to a particular type or kind of subject. In some embodiments, the subject is a human. In some embodiments, the subject has a chronic infection or cancer.

In some embodiments, the method further comprises administering to the subject a particular tyrosine kinase inhibitor. In some embodiments, the tyrosine kinase inhibitor is capable of inhibiting TCR signaling and/or CAR signaling. In some embodiments, the tyrosine kinase inhibitor is a Lck kinase inhibitor. In some embodiments, the tyrosine kinase inhibitor is a Fyn kinase inhibitor. In some embodiments, the tyrosine kinase inhibitor is a Src family tyrosine kinase inhibitor. In some embodiments, tyrosine kinase inhibitor is dasatinib or ponatinib.

In certain embodiments, the present invention provides for treating an immune system related condition or disease in a subject comprising administering to the subject genetically engineered T cells and a therapeutically effective amount of a pharmaceutical composition comprising a compound of the present invention (e.g., a compound having a thiazole, imidazolepyridiazine or piperazinyl-methyl-aniline structure).

In some embodiments, the treatment is prophylactic. In some embodiments, the pharmaceutical composition and the genetically engineered T cells are administered simultaneously and/or at different time points.

In some embodiments, the pharmaceutical compositions are administered iteratively for purposes of facilitating periods of T cell inactivation (e.g., during pharmaceutical composition administration) and periods of T cell activation (e.g., during absence of pharmaceutical composition administration; following clearance of the pharmaceutical composition).

Such methods are not limited to a specific type or kind of genetically engineered T cells. In some embodiments, the genetically engineered T cells include, but are not limited to, CAR T cells, genetically engineered TCR expressing T cells, genetically engineered T cells configured for tumor infiltrating lymphocyte (TIL) therapy, genetically engineered T cells configured for transduced T-cell therapy, and/or viral specific T cells reengineered with a TCR or CAR.

Such methods are not limited to treating a specific immune system related condition or disease. In some embodiments, the immune system related condition or disease is selected from cancer or an autoimmune disease or condition.

In some embodiments, the method further comprises administering to the subject a particular tyrosine kinase inhibitor. In some embodiments, the tyrosine kinase inhibitor is capable of inhibiting TCR signaling and/or CAR signaling. In some embodiments, the tyrosine kinase inhibitor is a Lck kinase inhibitor. In some embodiments, the tyrosine kinase inhibitor is a Fyn kinase inhibitor. In some embodiments, the tyrosine kinase inhibitor is a Src family tyrosine kinase inhibitor. In some embodiments, tyrosine kinase inhibitor is dasatinib or ponatinib.

In certain embodiments, the present invention provides methods for preventing and/or reversing toxicity related to genetically engineered T cell administered to a subject, comprising administering to the subject a therapeutically effective amount of a pharmaceutical composition comprising a compound of the present invention (e.g., a compound having a thiazole, imidazolepyridiazine or piperazinyl-methyl-aniline structure).

In some embodiments, such pharmaceutical compositions are administered iteratively for purposes of facilitating periods of T cell inactivation (e.g., during pharmaceutical composition administration) and periods of T cell activation (e.g., during absence of pharmaceutical composition administration; following clearance of the pharmaceutical composition).

Such methods are not limited to a specific type or kind of genetically engineered T cells. In some embodiments, the genetically engineered T cells include, but are not limited to, CAR T cells, genetically engineered TCR expressing T cells, genetically engineered T cells configured for tumor infiltrating lymphocyte (TIL) therapy, genetically engineered T cells configured for transduced T-cell therapy, and/or viral specific T cells reengineered with a TCR or CAR.

In some embodiments, the subject is undergoing an adoptive T cell therapy. Such methods are not limited to a particular type or kind of adoptive T cell therapy. In some embodiments, the adoptive T cell therapy is a CAR T-cell therapy. In some embodiments, the adoptive T cell therapy is a transduced T-cell therapy. In some embodiments, the adoptive T cell therapy is a tumor infiltrating lymphocyte (TIL) therapy.

Such methods are not limited to a particular type or kind of toxicity related to genetically engineered T cell administered to a subject. In some embodiments, the toxicity related to genetically engineered T cell administered to a subject is cytokine release syndrome. In some embodiments, the toxicity related to genetically engineered T cell administered to a subject is on-target off tumor toxicity or off-target off-tumor toxicity.

In some embodiments, the method further comprises administering to the subject a particular tyrosine kinase inhibitor. In some embodiments, the tyrosine kinase inhibitor is capable of inhibiting TCR signaling and/or CAR signaling. In some embodiments, the tyrosine kinase inhibitor is a Lck kinase inhibitor. In some embodiments, the tyrosine kinase inhibitor is a Fyn kinase inhibitor. In some embodiments, the tyrosine kinase inhibitor is a Src family tyrosine kinase inhibitor. In some embodiments, tyrosine kinase inhibitor is dasatinib or ponatinib.

In certain embodiments, the present invention provides compositions comprising a genetically engineered T cell population, wherein the genetically engineered T cell population was expanded in the presence of a compound of the present invention (e.g., a compound having a thiazole, imidazolepyridiazine or piperazinyl-methyl-aniline structure). In some embodiments, the compound is capable of increasing CAR-T cell expression of one or more of POLDIP2, GSTK1, and STMN2. In some embodiments, the compound is capable of decreasing CAR-T cell expression of one or more GZMB, MAPRE2, NAMPT, and SIGMAR1. In some embodiments, the compound is capable of inhibiting TCR signaling and/or CAR signaling.

In some embodiments, the genetically engineered T cell population is further expanded in the presence of a particular tyrosine kinase inhibitor. In some embodiments, the tyrosine kinase inhibitor is capable of inhibiting TCR signaling and/or CAR signaling. In some embodiments, the tyrosine kinase inhibitor is a Lck kinase inhibitor. In some embodiments, the tyrosine kinase inhibitor is a Fyn kinase inhibitor. In some embodiments, the tyrosine kinase inhibitor is a Src family tyrosine kinase inhibitor. In some embodiments, tyrosine kinase inhibitor is dasatinib or ponatinib.

In certain embodiments, the present invention provides methods of generating a population of genetically engineered T cells resistant to T cell exhaustion, comprising expanding a population of genetically engineered T cells in the presence of a compound of the present invention (e.g., a compound having a thiazole, imidazolepyridiazine or piperazinyl-methyl-aniline structure). In some embodiments, the compound is capable of increasing CAR-T cell expression of one or more of POLDIP2, GSTK1, and STMN2. In some embodiments, the compound is capable of decreasing CAR-T cell expression of one or more GZMB, MAPRE2, NAMPT, and SIGMAR1. In some embodiments, the compound is capable of inhibiting TCR signaling and/or CAR signaling inhibitor.

Such methods are not limited to a specific type or kind of genetically engineered T cells. In some embodiments, the genetically engineered T cells include, but are not limited to, CAR T cells, genetically engineered TCR expressing T cells, genetically engineered T cells configured for tumor infiltrating lymphocyte (TIL) therapy, genetically engineered T cells configured for transduced T-cell therapy, and/or viral specific T cells reengineered with a TCR or CAR. Such methods are not limited to a specific expanding technique as such techniques are well known in the art.

In some embodiments, the method further comprises expanding the genetically engineered T cell population in the presence of a particular tyrosine kinase inhibitor. In some embodiments, the tyrosine kinase inhibitor is capable of inhibiting TCR signaling and/or CAR signaling. In some embodiments, the tyrosine kinase inhibitor is a Lck kinase inhibitor. In some embodiments, the tyrosine kinase inhibitor is a Fyn kinase inhibitor. In some embodiments, the tyrosine kinase inhibitor is a Src family tyrosine kinase inhibitor. In some embodiments, tyrosine kinase inhibitor is dasatinib or ponatinib.

In certain embodiments, the present invention provides methods of treating an immune system related condition or disease in a subject undergoing an adoptive T cell therapy, comprising administering to the subject a genetically engineered T cell population that was expanded in the presence of a compound of the present invention (e.g., a compound having a thiazole, imidazolepyridiazine or piperazinyl-methyl-aniline structure). In some embodiments, the compound is capable of increasing CAR-T cell expression of one or more of POLDIP2, GSTK1, and STMN2. In some embodiments, the compound is capable of decreasing CAR-T cell expression of one or more GZMB, MAPRE2, NAMPT, and SIGMAR1. In some embodiments, the compound is capable of inhibiting TCR signaling and/or CAR signaling inhibitor. In some embodiments, the compound is capable of modulating (e.g., inhibiting) TCR or CAR-mediated signaling related to antigen-dependent or antigen-independent CAR T cell activation.

In some embodiments, the immune system related condition or disease is selected from cancer or an autoimmune disease or condition.

Such methods are not limited to a specific type or kind of genetically engineered T cells. In some embodiments, the genetically engineered T cells include, but are not limited to, CAR T cells, genetically engineered TCR expressing T cells, genetically engineered T cells configured for tumor infiltrating lymphocyte (TIL) therapy, genetically engineered T cells configured for transduced T-cell therapy, and/or viral specific T cells reengineered with a TCR or CAR.

Such methods are not limited to a particular type or kind of adoptive T cell therapy. In some embodiments, the adoptive T cell therapy is a CAR T-cell therapy. In some embodiments, the adoptive T cell therapy is a transduced T-cell therapy. In some embodiments, the adoptive T cell therapy is a tumor infiltrating lymphocyte (TIL) therapy.

In some embodiments, the method further comprises expanding the genetically engineered T cell population in the presence of a particular tyrosine kinase inhibitor. In some embodiments, the tyrosine kinase inhibitor is capable of inhibiting TCR signaling and/or CAR signaling. In some embodiments, the tyrosine kinase inhibitor is a Lck kinase inhibitor. In some embodiments, the tyrosine kinase inhibitor is a Fyn kinase inhibitor. In some embodiments, the tyrosine kinase inhibitor is a Src family tyrosine kinase inhibitor. In some embodiments, tyrosine kinase inhibitor is dasatinib or ponatinib.

The present invention contemplates that exposure of animals (e.g., humans) suffering from cancer (e.g., and/or cancer related disorders) to adoptive T cell therapies (e.g., a CAR T-cell therapy, a transduced T-cell therapy, and a tumor infiltrating lymphocyte (TIL) therapy) with genetically engineered T cell populations and pharmaceutical compositions comprising a compound of the present invention (e.g., a compound having a thiazole, imidazolepyridiazine or piperazinyl-methyl-aniline structure) will inhibit the growth of cancer cells or supporting cells outright and/or render such cells as a population more susceptible to the cell death-inducing activity of cancer therapeutic drugs or radiation therapies. In such embodiments, the methods result in improved therapy outcome as such pharmaceutical compositions are capable of 1) increasing CAR-T cell expression of one or more of POLDIP2, GSTK1, and STMN2; 2) decreasing CAR-T cell expression of one or more GZMB, MAPRE2, NAMPT, and SIGMAR1; 3) modulating TCR signaling within the genetically engineered T cell population (e.g., decreasing expression of one or more of PD-1, TIM-3, and LAG-3; increasing expression of memory markers (e.g., CD62L or CCR7); decreasing secretion of IL-2 and other cytokines; increasing secretion of IL-2 and other cytokines after transient pharmaceutical composition treatment and subsequent clearance of the pharmaceutical composition), 4) preventing and/or reversing T cell exhaustion within the genetically engineered T cell population; and 5) preventing and/or T cell exhaustion related to antigen-dependent or antigen-independent CAR T cell activation. Thus, the present invention provides methods for treating cancer (e.g., and/or cancer related disorders) with adoptive T cell therapies (e.g., a CAR T-cell therapy, a transduced T-cell therapy, and a tumor infiltrating lymphocyte (TIL) therapy) in a subject comprising administering to the subject (e.g., simultaneously and/or at different time points) genetically engineered T cells, particular pharmaceutical compositions comprising a compound of the present invention (e.g., a compound having a thiazole, imidazolepyridiazine or piperazinyl-methyl-aniline structure), and additional therapeutic agents (e.g., particular tyrosine kinase inhibitors (e.g., dasatinib, ponatinib), cancer therapeutic drugs or radiation therapies.

The present invention contemplates that exposure of animals (e.g., humans) suffering from cancer (e.g., and/or cancer related disorders) to adoptive T cell therapies (e.g., a CAR T-cell therapy, a transduced T-cell therapy, and a tumor infiltrating lymphocyte (TIL) therapy) with genetically engineered T cell populations that were expanded in the presence of particular compounds of the present invention (e.g., compounds having a thiazole, imidazolepyridiazine or piperazinyl-methyl-aniline structure) will inhibit the growth of cancer cells or supporting cells outright and/or render such cells as a population more susceptible to the cell death-inducing activity of cancer therapeutic drugs or radiation therapies. In such embodiments, the methods result in improved therapy outcome as such genetically engineered T cell populations are resistant and/or less prone to T cell exhaustion. Thus, the present invention provides methods for treating cancer (e.g., and/or cancer related disorders) with adoptive T cell therapies (e.g., a CAR T-cell therapy, a transduced T-cell therapy, and a tumor infiltrating lymphocyte (TIL) therapy) in a subject comprising administering to the subject (e.g., simultaneously and/or at different time points) genetically engineered T cell populations that were expanded in the presence of particular compounds of the present invention (e.g., compounds having a thiazole, imidazolepyridiazine or piperazinyl-methyl-aniline structure), and additional therapeutic agents (e.g., particular tyrosine kinase inhibitors (e.g., dasatinib, ponatinib), cancer therapeutic drugs or radiation therapies.

The present invention contemplates that such methods (e.g., adoptive T cell therapies with genetically engineered T cell populations and compositions comprising particular compounds of the present invention) (e.g., adoptive T cell therapies with genetically engineered T cell populations that were expanded in the presence of particular compounds of the present invention) satisfy an unmet need for the treatment of multiple cancer types, either when administered as monotherapy or when administered in a temporal relationship with additional agent(s), such as particular tyrosine kinase inhibitors (e.g., dasatinib, ponatinib), other cell death-inducing or cell cycle disrupting cancer therapeutic drugs or radiation therapies (combination therapies), so as to render a greater proportion of the cancer cells or supportive cells susceptible to executing the apoptosis program compared to the corresponding proportion of cells in an animal treated only with the cancer therapeutic drug or radiation therapy alone.

In certain embodiments of the invention, combination treatment of animals with such methods (e.g., adoptive T cell therapies with genetically engineered T cell populations and compositions comprising particular compounds of the present invention) (e.g., adoptive T cell therapies with genetically engineered T cell populations that were expanded in the presence of particular compounds of the present invention) produce a greater tumor response and clinical benefit in such animals compared to those treated with the anticancer drugs/radiation alone. Since the doses for all approved anticancer drugs and radiation treatments are known, the present invention contemplates the various combinations of them with such methods.

A non-limiting exemplary list of cancer (e.g., and/or cancer related disorders) includes, but is not limited to, pancreatic cancer, breast cancer, prostate cancer, lymphoma, skin cancer, colon cancer, melanoma, malignant melanoma, ovarian cancer, brain cancer, primary brain carcinoma, head and neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like, T and B cell mediated autoimmune diseases; inflammatory diseases; infections; hyperproliferative diseases; AIDS; degenerative conditions, vascular diseases, and the like. In some embodiments, the cancer cells being treated are metastatic. In other embodiments, the cancer cells being treated are resistant to anticancer agents.

### BRIEF DESCRIPTION OF THE DRAWINGS

FIG. 1: Functional characterization of new compounds experiment #1. A-B) CD19.28ζ CAR-T cells were grown with or without compounds for 24 hours. CAR-T cells were then co-cultured with Nalm6-GL leukemia cells at a 1:1 effector:target ratio for 6 hours, after which CD69 and CD107a surface expression was assessed via flow cytometry. A) Untreated control showing robust CD19.28ζ CAR-T CD69+/CD107a+ double positive cells (top right quadrant). B) Dose-titration of compounds at 10 nM, 20 nM, and 1 uM concentrations. Dasatinib was used as a positive control for inhibition of CAR-T cell activation.

FIG. 2: Functional characterization of new compounds experiment #2. A-B) CD19.28ζ CAR-T cells were grown with or without compounds for 24 hours. CAR-T cells were then co-cultured with Nalm6-GL leukemia cells at a 1:1 effector:target ratio for 6 hours, after which CD69 and CD107a surface expression was assessed via flow cytometry. A) Positive control showing robust CD19.28ζ CAR-T CD69+/CD107a+ double positive cells (top right quadrant). B) Dose-titration of compounds at 100 nM, 1 uM, and/or 10 uM concentrations. Cell viability was noted for compounds which exhibited toxicity, defined as viability <40% (red). Dasatinib was used as a positive control for inhibition of CAR-T cell activation.

FIG. 3: Functional characterization of new compounds experiment #3. A-B) CD19.28ζ CAR-T cells were grown with or without compounds for 24 hours. CAR-T cells were then co-cultured with Nalm6-GL leukemia cells at a 1:1 effector:target ratio for 6 hours, after which CD69 and CD107a surface expression was assessed via flow cytometry. A) Positive control showing robust CD19.28ζ CAR-T CD69+/CD107a+ double positive cells (top right quadrant). B) Dose-titration of compounds at 1 nM, 10 nM, 100 nM, 1 uM, 10 uM concentrations. Dasatinib and ponatinib were used as positive controls for inhibition of CAR-T cell activation.

FIG. 4: Functional characterization of new compounds experiment #4. A-B) CD19.28ζ CAR-T cells were grown with or without compounds for 24 hours. CAR-T cells were then co-cultured with Nalm6-GL leukemia cells at a 1:1 effector:target ratio for 6 hours, after which CD69 and CD107a surface expression was assessed via flow cytometry. A) Positive control showing robust CD19.28ζ CAR-T CD69+/CD107a+ double positive cells (top right quadrant). B) Dose-titration of compounds at 10 nM, 1 uM, and 10 uM concentrations. Dasatinib was used as a positive control for inhibition of CAR-T cell activation.

FIG. 5: Functional characterization of new compounds experiment #5. A-B) CD19.28ζ CAR-T cells were grown with or without compounds for 24 hours. CAR-T cells were then co-cultured with Nalm6-GL leukemia cells at a 1:1 effector:target ratio for 6 hours, after which CD69 and CD107a surface expression was assessed via flow cytometry. A) Positive control showing robust CD19.28ζ CAR-T CD69+/CD107a+ double positive cells (top right quadrant). B) Dose-titration of compounds at 10 nM, 1 uM, and 10 uM concentrations. Dasatinib was used as a positive control for inhibition of CAR-T cell activation.

FIG. 6: Summary results from experiments 1-5. A-B) Bar graph represents the mean+/−the standard error mean of CD69+/CD107a+ double positive CD19.28ζ CAR-T cells that were untreated or treated with compounds for 24 hours prior to co-culture with Nalm6-GL leukemia. Compounds that decreased CD69+/CD107a+ cells compared to untreated controls are shown here (n=1-5).

### DEFINITIONS

It must be noted that, as used in this specification and the appended claims, the singular forms “a,” “an” and “the” include plural referents unless the content clearly dictates otherwise. Thus, for example, reference to “a T cell” includes two or more T cells, and the like.

The term “about,” particularly in reference to a given quantity, is meant to encompass deviations of plus or minus five percent.

The term “chimeric antigen receptor” or “CAR,” as used herein, refers to an artificial T cell receptor that is engineered to be expressed on an immune effector cell and specifically bind an antigen. CARs may be used as a therapy with adoptive cell transfer. T cells are removed from a patient and modified so that they express the receptors specific to a particular form of antigen. In some embodiments, the CARs have been expressed with specificity to a tumor associated antigen, for example. CARs may also comprise an intracellular activation domain, a transmembrane domain and an extracellular domain comprising a tumor associated antigen binding region. The specificity of CAR designs may be derived from ligands of receptors (e.g., peptides). In some embodiments, a CAR can target cancers by redirecting the specificity of a T cell expressing the CAR specific for tumor associated antigens.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

The term “T cell” refers to T lymphocytes as defined in the art and is intended to include thymocytes, immature T lymphocytes, mature T lymphocytes, resting T lymphocytes, or activated T lymphocytes. The T cells can be CD4⁺ T cells, CD8⁺ T cells, CD4⁺CD8⁺ T cells, or CD4⁻CD8⁻ cells. The T cells can also be T helper cells, such as T helper 1 (TH1), or T helper 2 (TH2) cells, or TH17 cells, as well as cytotoxic T cells, regulatory T cells, natural killer T cells, naïve T cells, memory T cells, or gamma delta T cells.

The T cells can be a purified population of T cells, or alternatively the T cells can be in a population with cells of a different type, such as B cells and/or other peripheral blood cells. The T cells can be a purified population of a subset of T cells, such as CD4⁺ T cells, or they can be a population of T cells comprising different subsets of T cells. In another embodiment of the invention, the T cells are T cell clones that have been maintained in culture for extended periods of time. T cell clones can be transformed to different degrees. In a specific embodiment, the T cells are a T cell clone that proliferates indefinitely in culture.

In some embodiments, the T cells are primary T cells. The term “primary T cells” is intended to include T cells obtained from an individual, as opposed to T cells that have been maintained in culture for extended periods of time. Thus, primary T cells are particularly peripheral blood T cells obtained from a subject. A population of primary T cells can be composed of mostly one subset of T cells. Alternatively, the population of primary T cells can be composed of different subsets of T cells.

The T cells can be from previously stored blood samples, from a healthy individual, or alternatively from an individual affected with a condition. The condition can be an infectious disease, such as a condition resulting from a viral infection, a bacterial infection or an infection by any other microorganism, or a hyperproliferative disease, such as cancer like melanoma. In yet another embodiment of the invention, the T cells are from a subject suffering from or susceptible to an autoimmune disease or T-cell pathologies. The T cells can be of human origin, murine origin or any other mammalian species.

“T cell exhaustion” refers to loss of T cell function, which may occur as a result of an infection or a disease. T cell exhaustion is associated with increased expression of PD-1, TIM-3, and LAG-3, apoptosis, and reduced cytokine secretion.

By “therapeutically effective dose or amount” of an inhibitor of TCR signaling (e.g., a compound of the present invention (e.g., a compound having a thiazole, imidazolepyridiazine or piperazinyl-methyl-aniline structure)) is intended an amount that, when administered as described herein, brings about a positive therapeutic response in treatment of T cell exhaustion, such as restored T cell function. Improved T cell function may include increased T cell (e.g., CAR-T cell) expression of one or more of POLDIP2, GSTK1, and STMN2. Improved T cell function may include decreased CAR-T cell expression of one or more GZMB, MAPRE2, NAMPT, and SIGMAR1. Improved T cell function may include decreased expression of PD-1, TIM-3, and LAG-3, maintenance of memory markers (e.g., CD62L or CCR7), prevention of apoptosis, decreased secretion of IL-2 and other cytokines, increased secretion of IL-2 and other cytokines following transient treatment with such a compound and subsequent clearance of compound. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

The terms “subject,” “individual,” and “patient,” are used interchangeably herein and refer to any vertebrate subject, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

### DETAILED DESCRIPTION OF THE INVENTION

It has been shown that treatment with a particular tyrosine kinase inhibitor that inhibits T cell receptor signaling (e.g., a Lck tyrosine kinase inhibitor (e.g., dasatinib)) (e.g., a Src family tyrosine kinase inhibitor) reduced expression of the T cell exhaustion markers and improved formation of T cell memory (see, e.g., International Patent Application Publication No. 2018/183842). It has been shown that CAR T cells co-cultured with tumor cells in the presence of dasatinib or ponatinib exhibit attenuated activation and degranulation, fail to secrete cytokine, and display attenuated killing in response to tumor antigen (see, e.g., International Patent Application Publication No. 2018/183842). It has been shown that dasatinib potently inhibits the phosphorylation of CAR CD3z as well as distal signaling proteins after CAR crosslinking (see, e.g., International Patent Application Publication No. 2018/183842). It has been shown that tonically signaling CAR T cells expanded in the presence of dasatinib exhibit a reduction in canonical exhaustion marker expression in a dose-dependent manner, retain the capacity to form memory, display augmented cytokine secretion in response to tumor antigen, and display augmented cytotoxicity (see, e.g., International Patent Application Publication No. 2018/183842). It has been shown that in vivo dasatinib treatment suppresses exhaustion marker expression, augments memory formation, and facilitates cell survival/proliferation (see, e.g., International Patent Application Publication No. 2018/183842).

As indicated, experiments conducted during the course of developing embodiments for the present invention synthesized certain thiazole, imidazolepyridiazine and piperazinyl-methyl-aniline compounds and determined that such compounds function as modulators of CAR-T cell activity and effects related to CAR-T cell activity (e.g., preventing or reversing T cell exhaustion), and serve as therapeutics for use in CAR-T cell based therapies. For example, such experiments determined exposure of either a compound of the present invention or a tyrosine kinase inhibitor with healthy donor purified T cells that were artificially conditioned to become exhausted ex vivo by transducing them to express a CAR that tonically signals in the absence of antigen resulted in increased CAR-T cell expression of POLDIP2, GSTK1, and STMN2, and decreased CAR-T cell expression of GZMB, MAPRE2, NAMPT, and SIGMAR1. Moreover, additional experiments were conducted to assess the effects of the compounds recited herein on CAR T cell antigen-induced activation. Of the 27 compounds tested, 13 induced measurable suppression of CD69 and CD107a at the highest tested concentration of 10 uM, and 9 (EB1P083, EB1P084, EB1P085, EB1P086, EB1P088, EB1P089, EB1P090, EB1P091, EB2P067) induced measurable suppression at 1 uM. EB1P084, EB1P085, EB1P088, EB1P089, and EB2P067 exhibited the greatest potency at the 1 uM concentration compared to others.

Thus, the present invention relates to methods of preventing or reversing T cell exhaustion by exposing T cells experiencing T cell exhaustion to a new class of small-molecules having a thiazole, imidazolepyridiazine or piperazinyl-methyl-aniline structure, or by expanding genetically engineered T cells in the presence of such small molecules.

Certain thiazole, imidazolepyridiazine and piperazinyl-methyl-aniline compounds of the present invention may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

In a particular embodiment, thiazole compounds having Formula I, imidazolepyridiazine compounds having Formula II, and piperazinyl-methyl-aniline compounds having Formula III are provided as modulators of CAR-T cell activity and effects related to CAR-T cell activity (e.g., preventing or reversing T cell exhaustion):

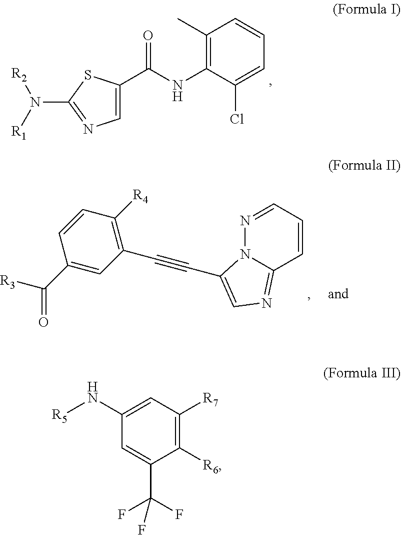

including pharmaceutically acceptable salts, solvates, and/or prodrugs thereof.

Formulas I, II and III are not limited to a particular chemical moiety for R1, R2, R3, R4, R5, R6 and R7. In some embodiments, the particular chemical moiety for R1, R2, R3, R4, R5, R6 and R7 independently include any chemical moiety that permits the resulting compound to increase CAR-T cell expression of one or more of POLDIP2, GSTK1, and STMN2. In some embodiments, the particular chemical moiety for R1, R2, R3, R4, R5, R6 and R7 independently include any chemical moiety that permits the resulting compound to decrease CAR-T cell expression of one or more of GZMB, MAPRE2, NAMPT, and SIGMAR1. In some embodiments, the particular chemical moiety for R1, R2, R3, R4, R5, R6 and R7 independently include any chemical moiety that permits the resulting compound to modulate (e.g., inhibit) CAR-T cell activity. In some embodiments, the particular chemical moiety for R1, R2, R3, R4, R5, R6 and R7 independently include any chemical moiety that permits the resulting compound to modulate (e.g., inhibit) TCR or CAR-mediated signaling related to antigen-dependent or antigen-independent CAR T cell activation. In some embodiments, the particular chemical moiety for R1, R2, R3, R4, R5, R6 and R7 independently include any chemical moiety that permits the resulting compound to prevent and/or reverse T cell exhaustion related to antigen-dependent or antigen-independent CAR T cell activation. In some embodiments, the particular chemical moiety for R1, R2, R3, R4, R5, R6 and R7 independently include any chemical moiety that permits the resulting compound to decrease CAR-T cell expression of one or more of PD-1, TIM-3, and LAG-3. In some embodiments, the particular chemical moiety for R1, R2, R3, R4, R5, R6 and R7 independently include any chemical moiety that permits the resulting compound to increase CAR-T cell expression of memory markers (e.g., CD62L). In some embodiments, the particular chemical moiety for R1, R2, R3, R4, R5, R6 and R7 independently include any chemical moiety that permits the resulting compound to prevent CAR-T cell apoptosis. In some embodiments, the particular chemical moiety for R1, R2, R3, R4, R5, R6 and R7 independently include any chemical moiety that permits the resulting compound to decrease CAR-T cell secretion of IL-2 and other cytokines. In some embodiments, the particular chemical moiety for R1, R2, R3, R4, R5, R6 and R7 independently include any chemical moiety that permits the resulting compound to decrease CAR-T cell secretion of IL-2 and other cytokines. In some embodiments, the particular chemical moiety for R1, R2, R3, R4, R5, R6 and R7 independently include any chemical moiety that permits the resulting compound to increase CAR-T cell secretion of IL-2 and other cytokines following transient treatment with such a compound and subsequent clearance of compound.

In some embodiments, R1 and R2 are independently selected from hydrogen,

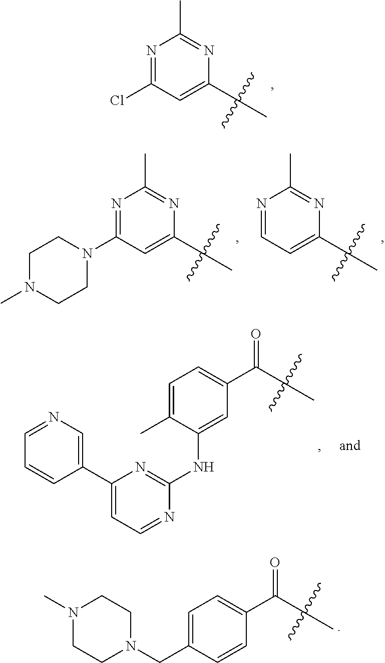

In some embodiments, R3 is selected from hydrogen, hydroxyl,

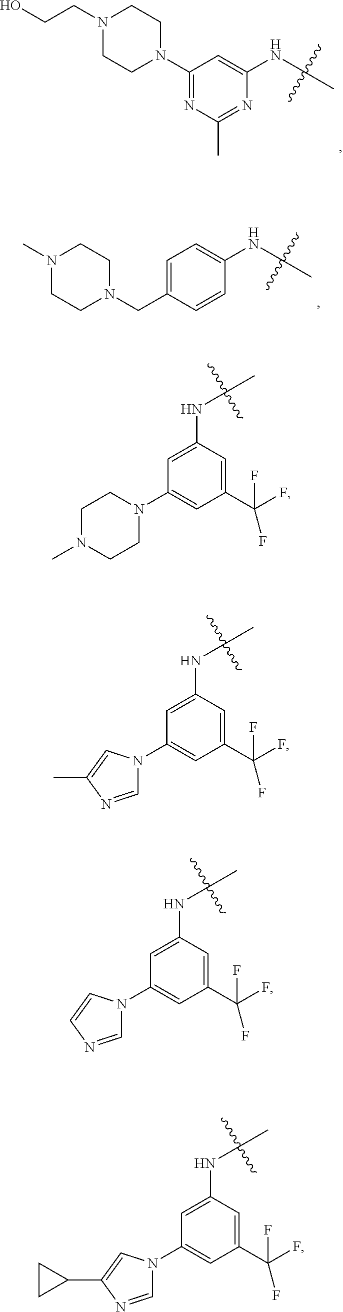 [SMILES: *Nc1cc(N2CCN(CCO)CC2)nc(C)n1 ; *Nc1ccc(CN2CCN(C)CC2)cc1 ; *Nc1cc(N2CCN(C)CC2)cc(C(F)(F)F)c1 ; *Nc1cc(-n2cnc(C)c2)cc(C(F)(F)F)c1 ; *Nc1cc(-n2ccnc2)cc(C(F)(F)F)c1 ; *Nc1cc(-n2cnc(C3CC3)c2)cc(C(F)(F)F)c1]

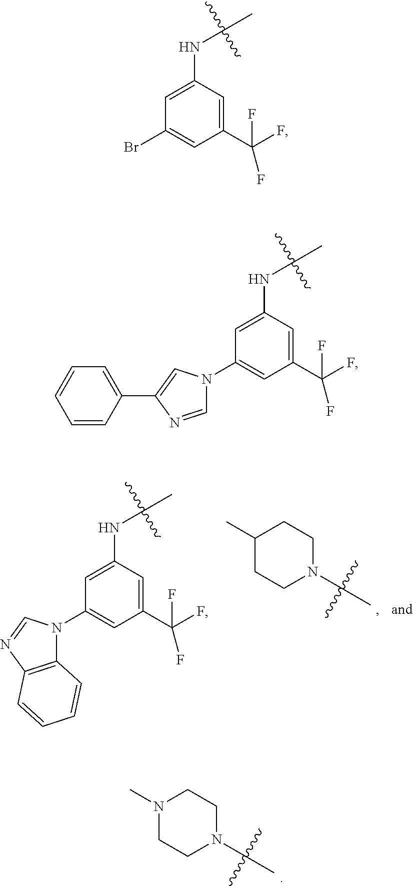

In some embodiments, R4 is hydrogen, methyl or

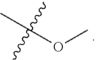 [SMILES: *OC]

In some embodiments, R5 is selected from hydrogen

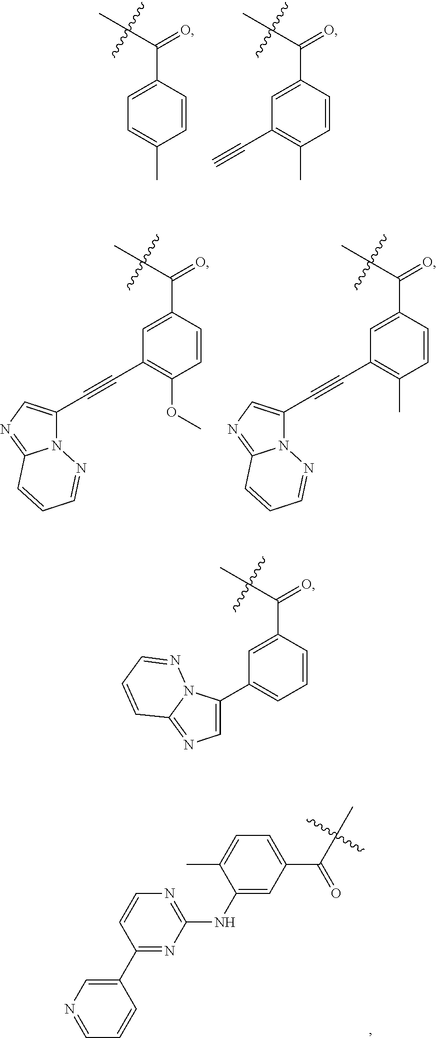

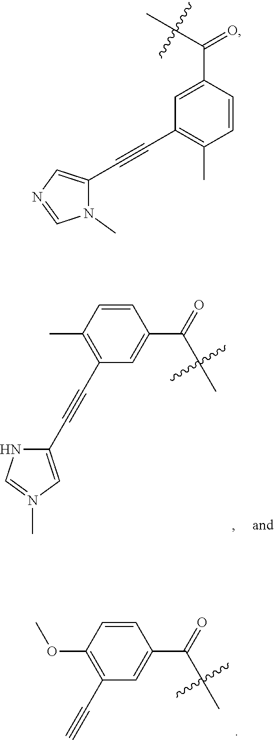

In some embodiments, R6 is hydrogen or

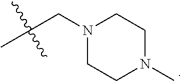 [SMILES: *CN1CCN(C)CC1]

In some embodiments, R7 is hydrogen of

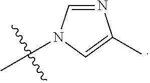 [SMILES: *n1cnc(C)c1]

In some embodiments, the following thiazole, imidazolepyridiazine and piperazinyl-methyl-aniline compounds are contemplated for Formulas I, II and III:

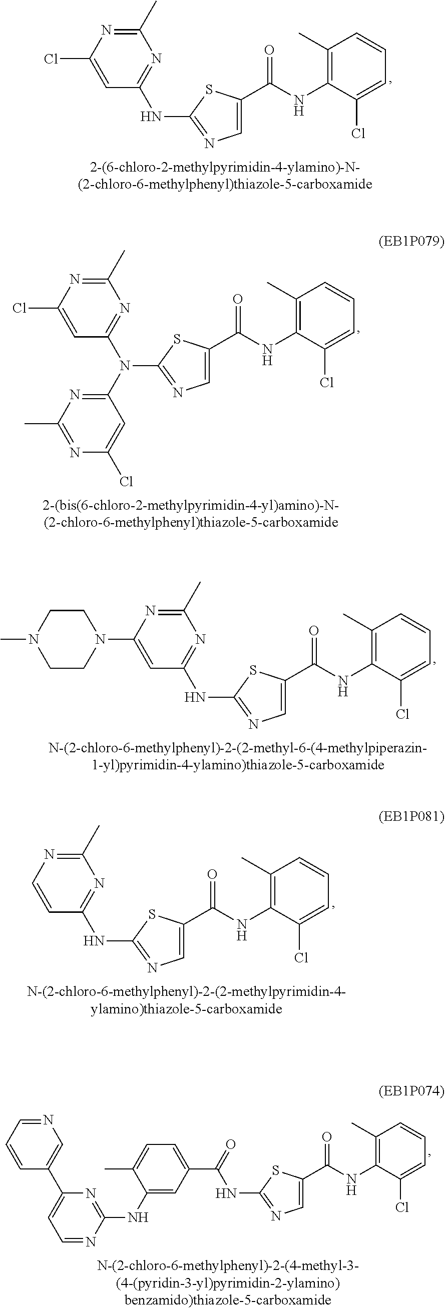 [SMILES: Cc1nc(Cl)cc(Nc2ncc(C(=O)Nc3c(C)cccc3Cl)s2)n1 ; Cc1nc(Cl)cc(N(c2cc(Cl)nc(C)n2)c2ncc(C(=O)Nc3c(C)cccc3Cl)s2)n1 ; Cc1nc(Nc2ncc(C(=O)Nc3c(C)cccc3Cl)s2)cc(N2CCN(C)CC2)n1 ; Cc1nccc(Nc2ncc(C(=O)Nc3c(C)cccc3Cl)s2)n1 ; Cc1ccc(C(=O)Nc2ncc(C(=O)Nc3c(C)cccc3Cl)s2)cc1Nc1nccc(-c2cccnc2)n1]

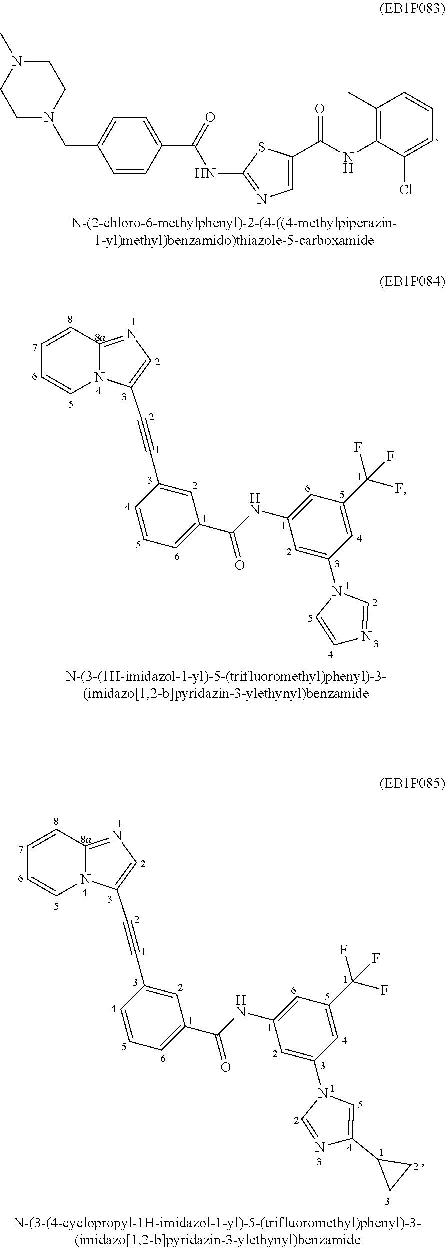 [SMILES: Cc1cccc(Cl)c1NC(=O)c1cnc(NC(=O)c2ccc(CN3CCN(C)CC3)cc2)s1 ; O=C(Nc1cc(-n2ccnc2)cc(C(F)(F)F)c1)c1cccc(C#Cc2cnc3ccccn23)c1 ; O=C(Nc1cc(-n2cnc(C3CC3)c2)cc(C(F)(F)F)c1)c1cccc(C#Cc2cnc3ccccn23)c1]

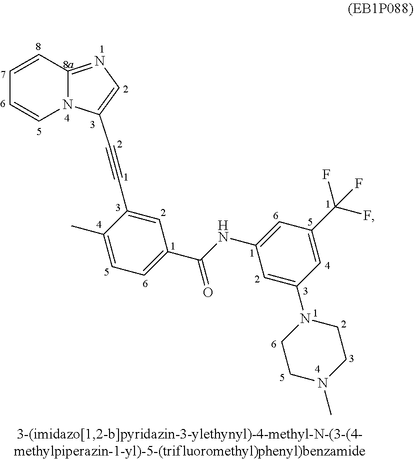 [SMILES: Cc1ccc(C(=O)Nc2cc(N3CCN(C)CC3)cc(C(F)(F)F)c2)cc1C#Cc1cnc2ccccn12]

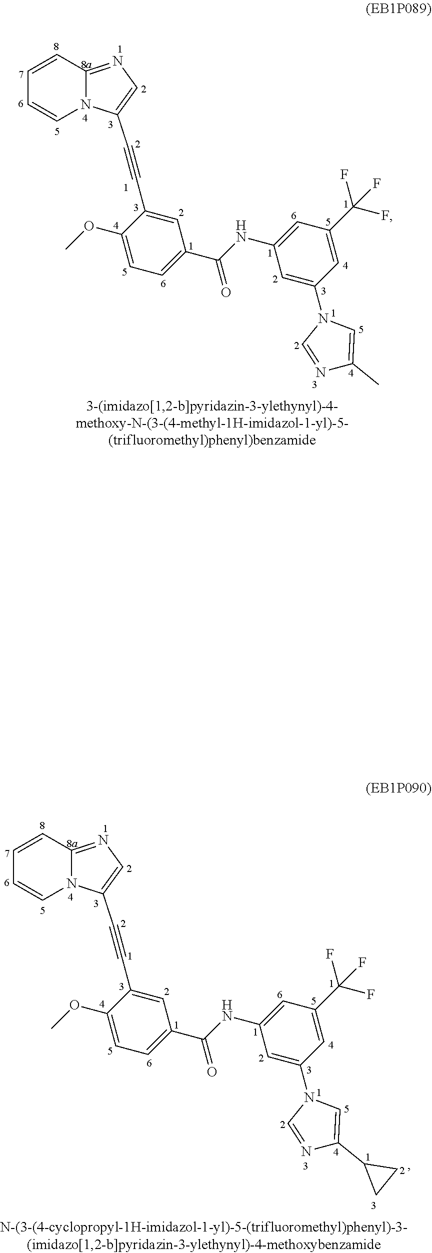 [SMILES: COc1ccc(C(=O)Nc2cc(-n3cnc(C)c3)cc(C(F)(F)F)c2)cc1C#Cc1cnc2ccccn12 ; COc1ccc(C(=O)Nc2cc(-n3cnc(C4CC4)c3)cc(C(F)(F)F)c2)cc1C#Cc1cnc2ccccn12]

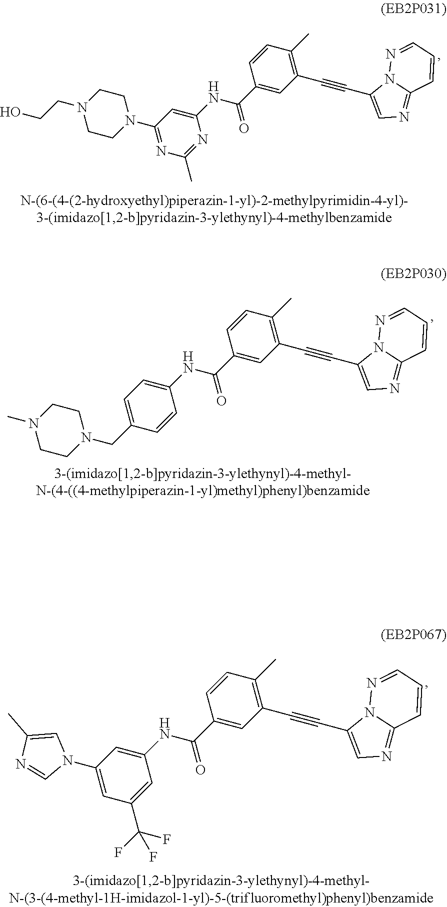 [SMILES: Cc1nc(NC(=O)c2ccc(C)c(C#Cc3cnc4cccnn34)c2)cc(N2CCN(CCO)CC2)n1 ; Cc1ccc(C(=O)Nc2ccc(CN3CCN(C)CC3)cc2)cc1C#Cc1cnc2cccnn12 ; Cc1cn(-c2cc(NC(=O)c3ccc(C)c(C#Cc4cnc5cccnn45)c3)cc(C(F)(F)F)c2)cn1]

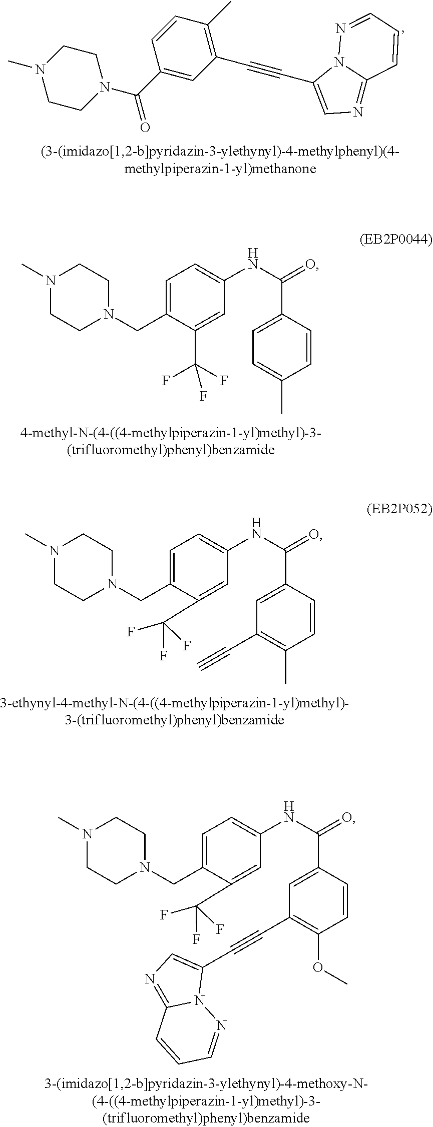

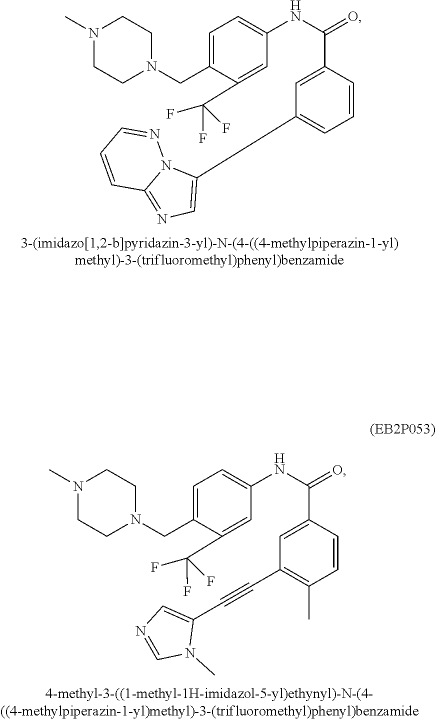 [SMILES: CN1CCN(Cc2ccc(NC(=O)c3cccc(-c4cnc5cccnn45)c3)cc2C(F)(F)F)CC1 ; Cc1ccc(C(=O)Nc2ccc(CN3CCN(C)CC3)c(C(F)(F)F)c2)cc1C#Cc1cncn1C]

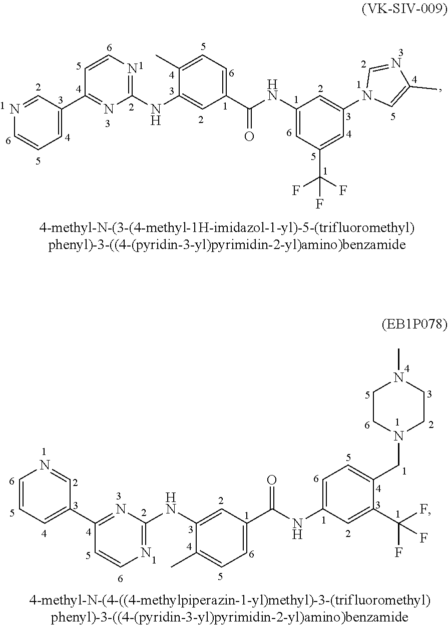 [SMILES: Cc1cn(-c2cc(NC(=O)c3ccc(C)c(Nc4nccc(-c5cccnc5)n4)c3)cc(C(F)(F)F)c2)cn1 ; Cc1ccc(C(=O)Nc2ccc(CN3CCN(C)CC3)c(C(F)(F)F)c2)cc1Nc1nccc(-c2cccnc2)n1]

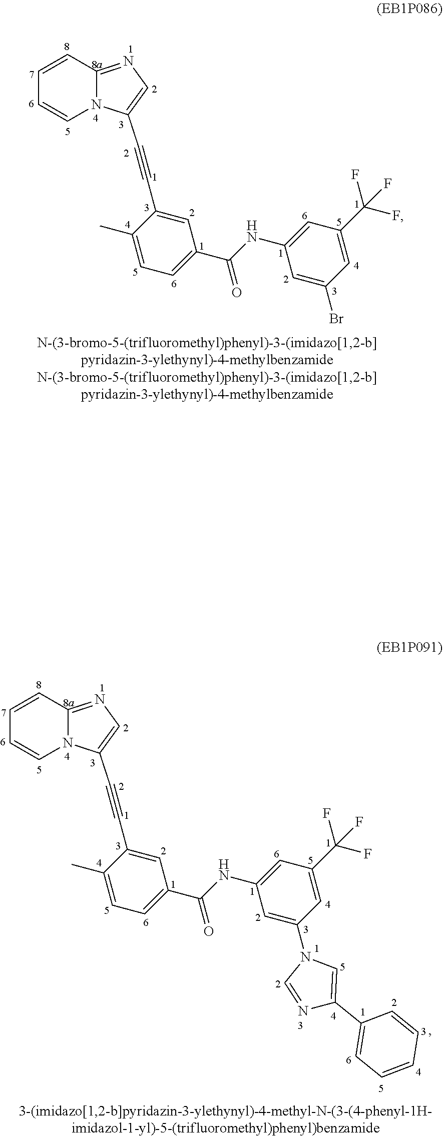 [SMILES: Cc1ccc(C(=O)Nc2cc(Br)cc(C(F)(F)F)c2)cc1C#Cc1cnc2ccccn12 ; Cc1ccc(C(=O)Nc2cc(-n3cnc(-c4ccccc4)c3)cc(C(F)(F)F)c2)cc1C#Cc1cnc2ccccn12]

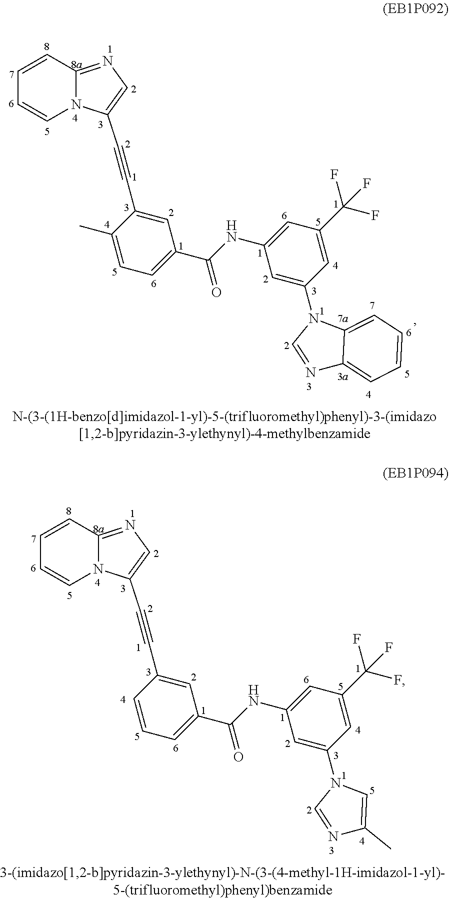 [SMILES: Cc1ccc(C(=O)Nc2cc(-n3cnc4ccccc43)cc(C(F)(F)F)c2)cc1C#Cc1cnc2ccccn12 ; Cc1cn(-c2cc(NC(=O)c3cccc(C#Cc4cnc5ccccn45)c3)cc(C(F)(F)F)c2)cn1]

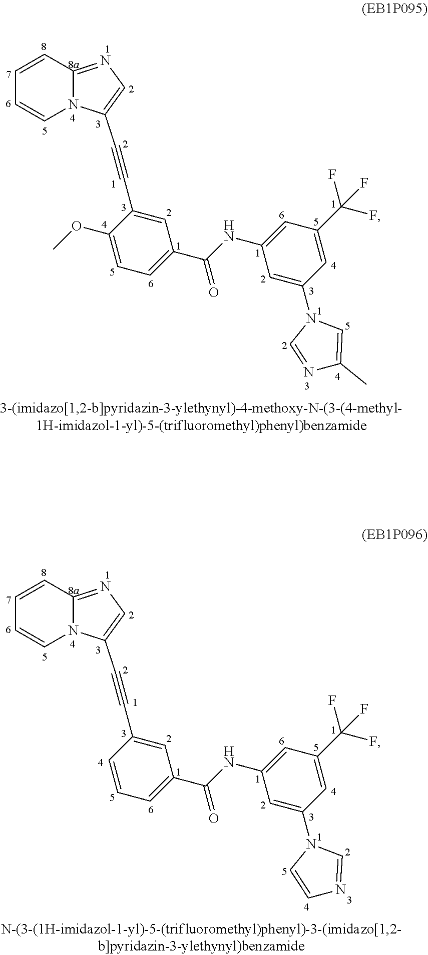 [SMILES: COc1ccc(C(=O)Nc2cc(-n3cnc(C)c3)cc(C(F)(F)F)c2)cc1C#Cc1cnc2ccccn12 ; O=C(Nc1cc(-n2ccnc2)cc(C(F)(F)F)c1)c1cccc(C#Cc2cnc3ccccn23)c1]

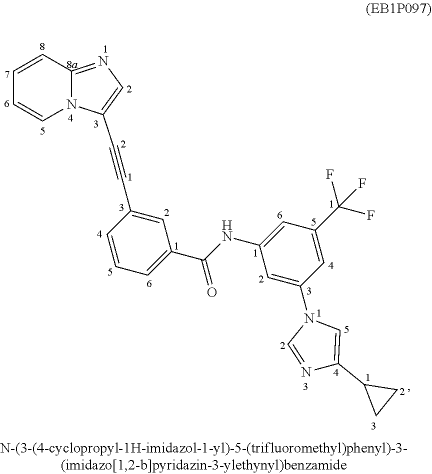 [SMILES: O=C(Nc1cc(-n2cnc(C3CC3)c2)cc(C(F)(F)F)c1)c1cccc(C#Cc2cnc3ccccn23)c1]

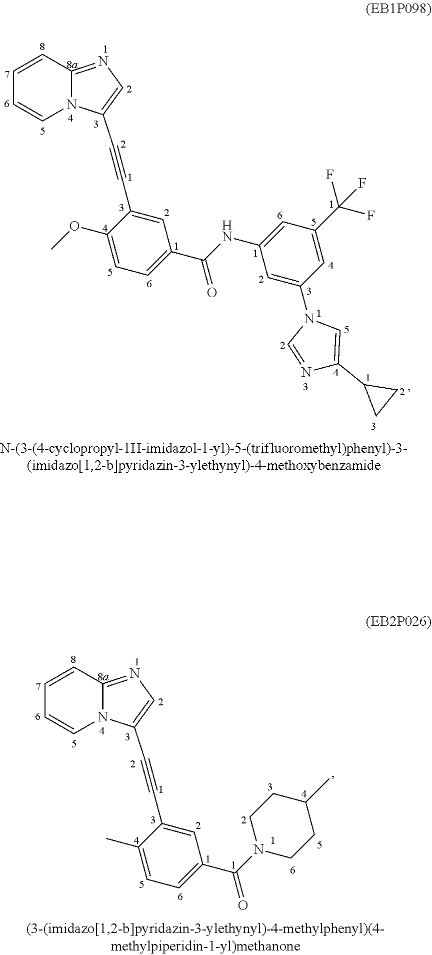 [SMILES: COc1ccc(C(=O)Nc2cc(-n3cnc(C4CC4)c3)cc(C(F)(F)F)c2)cc1C#Cc1cnc2ccccn12 ; Cc1ccc(C(=O)N2CCC(C)CC2)cc1C#Cc1cnc2ccccn12]

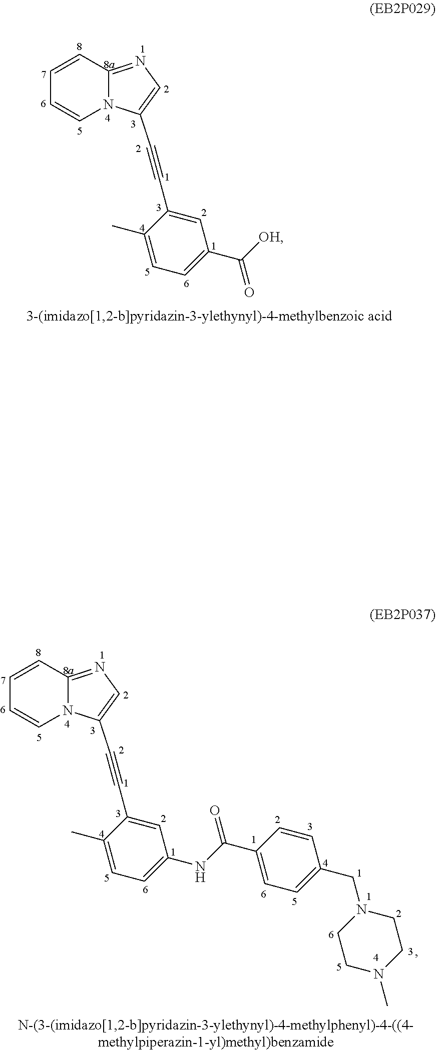 [SMILES: Cc1ccc(C(=O)O)cc1C#Cc1cnc2ccccn12 ; Cc1ccc(NC(=O)c2ccc(CN3CCN(C)CC3)cc2)cc1C#Cc1cnc2ccccn12]

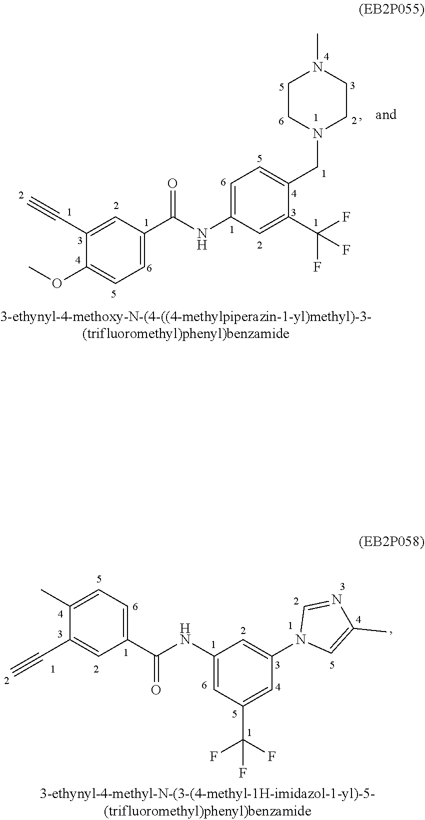 [SMILES: C#Cc1cc(C(=O)Nc2ccc(CN3CCN(C)CC3)c(C(F)(F)F)c2)ccc1OC ; C#Cc1cc(C(=O)Nc2cc(-n3cnc(C)c3)cc(C(F)(F)F)c2)ccc1C]

including pharmaceutically acceptable salts, solvates, and/or prodrugs thereof.

The invention further provides processes for preparing any of the compounds of the present invention through following any technique known to those of skill in a related art.

Accordingly, the present invention provides compositions and methods for preventing or reversing T cell exhaustion. In certain embodiments, the present invention relates to methods of preventing or reversing T cell exhaustion by exposing T cells experiencing T cell exhaustion to a new class of small-molecules having a thiazole, imidazolepyridiazine or piperazinyl-methyl-aniline structure, or by expanding genetically engineered T cells in the presence of such small molecules.

Indeed, the present invention contemplates that exposure of animals (e.g., humans) undergoing adoptive T cell therapies (e.g., a CAR T-cell therapy, a transduced T-cell therapy, and a tumor infiltrating lymphocyte (TIL) therapy) with genetically engineered T cell populations to compositions comprising particular compounds of the present invention will result in improved therapy outcome as such particular compounds are capable of 1) increasing CAR-T cell expression of one or more of POLDIP2, GSTK1, and STMN2; 2) decreasing CAR-T cell expression of one or more GZMB, MAPRE2, NAMPT, and SIGMAR1; 3) modulating TCR signaling within the genetically engineered T cell population (e.g., decreasing expression of one or more of PD-1, TIM-3, and LAG-3; increasing expression of memory markers (e.g., CD62L or CCR7); decreasing secretion of IL-2 and other cytokines; increasing secretion of IL-2 and other cytokines following transient treatment with such a composition and subsequent clearance of the composition), 4) preventing and/or reversing T cell exhaustion within the genetically engineered T cell population, 5) preventing and/or reversing T cell exhaustion related to antigen-dependent or antigen-independent CAR T cell activation.

Thus, the present invention provides methods for treating an immune system related condition or disease (e.g., cancer) in a subject comprising administering to the subject (e.g., simultaneously and/or at different time points) genetically engineered T cells and particular compounds of the present invention.

In some embodiments, such particular compounds are administered iteratively for purposes of facilitating periods of T cell inactivation (e.g., during compound administration) and periods of T cell activation (e.g., during absence of compound administration; following clearance of the compound).

Such methods are not limited to a specific type or kind of genetically engineered T cells. In some embodiments, the genetically engineered T cells include, but are not limited to, CAR T cells, genetically engineered TCR expressing T cells, genetically engineered T cells configured for tumor infiltrating lymphocyte (TIL) therapy, genetically engineered T cells configured for transduced T-cell therapy, and/or viral specific T cells reengineered with a TCR or CAR.

In some embodiments, the methods further comprise administering to the subject a particular tyrosine kinase inhibitor. In some embodiments, the tyrosine kinase inhibitor is capable of inhibiting TCR signaling and/or CAR signaling. In some embodiments, the tyrosine kinase inhibitor is a Lck kinase inhibitor. In some embodiments, the tyrosine kinase inhibitor is a Fyn kinase inhibitor. In some embodiments, the tyrosine kinase inhibitor is a Src family tyrosine kinase inhibitor. In some embodiments, tyrosine kinase inhibitor is dasatinib or ponatinib.

Such compounds may be administered by any suitable mode of administration, but are typically administered orally. Multiple cycles of treatment may be administered to a subject. In certain embodiments, the compounds are administered according to a daily dosing regimen or intermittently.

In another embodiment, the compounds are administered for a period of time sufficient to restore at least partial T cell function, then discontinued. For example, in some embodiments, such compounds are administered iteratively for purposes of facilitating periods of T cell inactivation (e.g., during compound administration) and periods of T cell activation (e.g., during absence of compound administration; following clearance of the compound).

The present invention contemplates that ex vivo expansion of a population of T cells with particular compounds of the present invention will result in a population T cells that are resistant and/or less prone to T cell exhaustion. Thus, the present invention provides compositions comprising a population of T cells that were expanded in the presence of particular compounds of the present invention. Thus, the present invention provides methods of expanding a population of T cells to generate T cell populations that are resistant and/or less prone to T cell exhaustion through expanding such T cells in the presence of particular compounds of the present invention. Thus, the present invention provides kits comprising T cell populations that were expanded in the presence particular compounds of the present invention and additional agents (e.g., additional agents useful in expanding T cells) (e.g., additional agents useful in adoptive T cell therapies (e.g., a CAR T-cell therapy, a transduced T-cell therapy, and a tumor infiltrating lymphocyte (TIL) therapy). Such methods are not limited to a specific type or kind of genetically engineered T cells. In some embodiments, the genetically engineered T cells include, but are not limited to, CAR T cells, genetically engineered TCR expressing T cells, genetically engineered T cells configured for tumor infiltrating lymphocyte (TIL) therapy, genetically engineered T cells configured for transduced T-cell therapy, and/or viral specific T cells reengineered with a TCR or CAR.

In some embodiments, the T cells are further expanded in the presence of a particular tyrosine kinase inhibitor. In some embodiments, the tyrosine kinase inhibitor is capable of inhibiting TCR signaling and/or CAR signaling. In some embodiments, the tyrosine kinase inhibitor is a Lck kinase inhibitor. In some embodiments, the tyrosine kinase inhibitor is a Fyn kinase inhibitor. In some embodiments, the tyrosine kinase inhibitor is a Src family tyrosine kinase inhibitor. In some embodiments, tyrosine kinase inhibitor is dasatinib or ponatinib.

The present invention contemplates that ex vivo expansion of a population of genetically engineered T cells (e.g., genetically engineered for use within adoptive T cell therapies (e.g., a CAR T-cell therapy, a transduced T-cell therapy, and a tumor infiltrating lymphocyte (TIL) therapy)) with particular compounds of the present invention will result in genetically engineered T cells that are resistant and/or less prone to T cell exhaustion. Thus, the present invention provides compositions comprising a population of genetically engineered T cells that were expanded in the presence of particular compounds of the present invention. Thus, the present invention provides methods of expanding a population of genetically engineered T cells to generate genetically engineered T cell populations that are resistant and/or less prone to T cell exhaustion through expanding such T cells in the presence of particular compounds of the present invention. Thus, the present invention provides kits comprising genetically engineered T cell populations that were expanded in the presence of particular compounds of the present invention. Such methods are not limited to a specific type or kind of genetically engineered T cells. In some embodiments, the genetically engineered T cells include, but are not limited to, CAR T cells, genetically engineered TCR expressing T cells, genetically engineered T cells configured for tumor infiltrating lymphocyte (TIL) therapy, genetically engineered T cells configured for transduced T-cell therapy, and/or viral specific T cells reengineered with a TCR or CAR.

In some embodiments, the genetically engineered T cell population is further expanded in the presence of a particular tyrosine kinase inhibitor. In some embodiments, the tyrosine kinase inhibitor is capable of inhibiting TCR signaling and/or CAR signaling. In some embodiments, the tyrosine kinase inhibitor is a Lck kinase inhibitor. In some embodiments, the tyrosine kinase inhibitor is a Fyn kinase inhibitor. In some embodiments, the tyrosine kinase inhibitor is a Src family tyrosine kinase inhibitor. In some embodiments, tyrosine kinase inhibitor is dasatinib or ponatinib.

The present invention contemplates that exposure of animals (e.g., humans) undergoing adoptive T cell therapies (e.g., a CAR T-cell therapy, a transduced T-cell therapy, and a tumor infiltrating lymphocyte (TIL) therapy) with genetically engineered T cell populations that were expanded in the presence of particular compounds of the present invention will result in improved therapy outcome as such genetically engineered T cell populations are resistant and/or less prone to T cell exhaustion. Thus, the present invention provides methods of treating an immune system related condition or disease (e.g., cancer) in a subject comprising administering a population of genetically engineered T cells expanded in the presence of particular compounds of the present invention. Such methods are not limited to a specific type or kind of genetically engineered T cells. In some embodiments, the genetically engineered T cells include, but are not limited to, CAR T cells, genetically engineered TCR expressing T cells, genetically engineered T cells configured for tumor infiltrating lymphocyte (TIL) therapy, genetically engineered T cells configured for transduced T-cell therapy, and/or viral specific T cells reengineered with a TCR or CAR.

In some embodiments, the genetically engineered T cell population is further expanded in the presence of a particular tyrosine kinase inhibitor. In some embodiments, the tyrosine kinase inhibitor is capable of inhibiting TCR signaling and/or CAR signaling. In some embodiments, the tyrosine kinase inhibitor is a Lck kinase inhibitor. In some embodiments, the tyrosine kinase inhibitor is a Fyn kinase inhibitor. In some embodiments, the tyrosine kinase inhibitor is a Src family tyrosine kinase inhibitor. In some embodiments, tyrosine kinase inhibitor is dasatinib or ponatinib.

Such embodiments are not limited to a particular type or kind of an immune system related condition or disease.

For example, in some embodiments, the immune system related condition or disease is an autoimmune disease or condition (e.g., Acquired Immunodeficiency Syndrome (AIDS), graft-versus-host disease (GVHD), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo, Wegener's granulomatosis, and any combination thereof).

For example, in some embodiments, the immune system related condition or disease is cancer (e.g., breast cancer, prostate cancer, ovarian cancer, cervical cancer, skin cancer, pancreatic cancer, colorectal cancer, renal cancer, liver cancer, brain cancer, lymphoma, leukemia, lung cancer, and thyroid carcinoma).

The present invention contemplates that the use of genetically engineered T cell populations that were expanded in the presence of particular compounds of the present invention within adoptive T cell therapies (e.g., a CAR T-cell therapy, a transduced T-cell therapy, and a tumor infiltrating lymphocyte (TIL) therapy) satisfies an unmet need as such therapies are frequently compromised by such T cell populations experiencing T cell exhaustion. Such methods are not limited to a specific type or kind of genetically engineered T cells. In some embodiments, the genetically engineered T cells include, but are not limited to, CAR T cells, genetically engineered TCR expressing T cells, genetically engineered T cells configured for tumor infiltrating lymphocyte (TIL) therapy, genetically engineered T cells configured for transduced T-cell therapy, and/or viral specific T cells reengineered with a TCR or CAR.

In some embodiments, the genetically engineered T cell population is further expanded in the presence of a particular tyrosine kinase inhibitor. In some embodiments, the tyrosine kinase inhibitor is capable of inhibiting TCR signaling and/or CAR signaling. In some embodiments, the tyrosine kinase inhibitor is a Lck kinase inhibitor. In some embodiments, the tyrosine kinase inhibitor is a Fyn kinase inhibitor. In some embodiments, the tyrosine kinase inhibitor is a Src family tyrosine kinase inhibitor. In some embodiments, tyrosine kinase inhibitor is dasatinib or ponatinib.

Some embodiments of the present invention provide for administering such methods (e.g., adoptive T cell therapies with genetically engineered T cell populations and compositions comprising particular compounds of the present invention) (e.g., adoptive T cell therapies with genetically engineered T cell populations that were expanded in the presence of particular compounds of the present invention) in combination with an effective amount of at least one additional therapeutic agent (including, but not limited to, particular tyrosine kinase inhibitors (e.g., dasatinib or ponatinib), chemotherapeutic antineoplastics, apoptosis-modulating agents, antimicrobials, antivirals, antifungals, and anti-inflammatory agents) and/or therapeutic technique (e.g., surgical intervention, and/or radiotherapies). In a particular embodiment, the additional therapeutic agent(s) is an anticancer agent.

The compounds of the present invention can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the pharmaceutical composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the compound of the present invention (e.g., when contained in a drug delivery system) in the pharmaceutical composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the pharmaceutical composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

The pharmaceutical compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the pharmaceutical compositions comprising one or more tyrosine kinase inhibitors (e.g., dasatinib, ponatinib) described herein are in unit dosage form, meaning an amount of a conjugate or composition of the invention appropriate for a single dose, in a premeasured or pre-packaged form.

The pharmaceutical compositions herein may optionally include one or more additional agents, or may be combined with one or more additional agents, such as other drugs for treating T cell exhaustion (e.g., anti-PD-1 checkpoint inhibitor, such as nivolumab), or other medications used to treat a subject for an infection or disease associated with T cell exhaustion (e.g., antiviral, antibiotic, or anti-cancer drugs and therapies, including adoptive T cell therapies). Compounded preparations may be used including at least one compound of the present invention and one or more other agents, such as other drugs for treating T cell exhaustion or an infection or disease associated with T cell exhaustion (e.g., tyrosine kinase inhibitors (e.g., dasatinib, ponatinib). Alternatively, such agents can be contained in a separate composition from the composition comprising a compound of the present invention and co-administered concurrently, before, or after the composition comprising a compound of the present invention.

At least one therapeutically effective cycle of treatment with a compound of the present invention will be administered to a subject for treatment of T cell exhaustion. By “therapeutically effective cycle of treatment” is intended a cycle of treatment that when administered, brings about a positive therapeutic response with respect to treatment of an individual for T cell exhaustion. Of particular interest is a cycle of treatment with a compound of the present invention that, when administered transiently as described herein, restores T cell function. For example, a therapeutically effective dose or amount of a compound of the present invention may increase CAR-T cell expression of one or more of POLDIP2, GSTK1, and STMN2, decrease CAR-T cell expression of one or more GZMB, MAPRE2, NAMPT, and SIGMAR1, decrease expression of PD-1, TIM-3, and LAG-3, improve maintenance of memory markers (e.g., CD62L or CCR7), prevent apoptosis, decrease secretion of IL-2 and other cytokines, and increase secretion of IL-2 and other cytokines following transient treatment with such a compound and subsequent clearance of compound.

In certain embodiments, multiple therapeutically effective doses of pharmaceutical compositions comprising one or more compounds of the present invention, and/or one or more other therapeutic agents, such as other drugs for treating T cell exhaustion (e.g., tyrosine kinase inhibitors (e.g., dasatinib, ponatinib) (e.g., anti-PD-1 checkpoint inhibitor, such as nivolumab), or other medications used to treat a subject for an infection or disease associated with T cell exhaustion (e.g., antiviral, antibiotic, or anti-cancer drugs and therapies, including adoptive T cell therapies) will be administered. The pharmaceutical compositions of the present invention are typically, although not necessarily, administered orally, via injection (subcutaneously, intravenously, or intramuscularly), by infusion, or locally. Additional modes of administration are also contemplated, such as topical, intralesion, intracerebral, intracerebroventricular, intraparenchymatous, pulmonary, rectal, transdermal, transmucosal, intrathecal, pericardial, intra-arterial, intraocular, intraperitoneal, and so forth.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising one or more compounds of the present invention and other agents may be administered using the same or different routes of administration in accordance with any medically acceptable method known in the art.

In another embodiment, the pharmaceutical compositions comprising one or more compounds of the present invention and/or other agents are administered prophylactically, e.g., to prevent T cell exhaustion. Such prophylactic uses will be of particular value for subjects with a chronic infection or cancer, who are at risk of developing T cell exhaustion.

In another embodiment of the invention, the pharmaceutical compositions comprising one or more compounds of the present invention and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The invention also provides a method for administering a conjugate comprising a compound of the present invention as provided herein to a patient suffering from a condition that is responsive to treatment with a compound of the present invention contained in the conjugate or composition. The method comprises administering, via any of the herein described modes, a therapeutically effective amount of the conjugate or drug delivery system, preferably provided as part of a pharmaceutical composition. The method of administering may be used to treat any condition that is responsive to treatment with compound of the present invention. More specifically, the pharmaceutical compositions herein are effective in treating T cell exhaustion.

Those of ordinary skill in the art will appreciate which conditions a compound of the present invention can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

Generally, a therapeutically effective amount will range from about 0.50 mg to 5 grams of a compound of the present invention daily, more preferably from about 5 mg to 2 grams daily, even more preferably from about 7 mg to 1.5 grams daily. Preferably, such doses are in the range of 10-600 mg four times a day (QID), 200-500 mg QID, 25-600 mg three times a day (TID), 25-50 mg TID, 50-100 mg TID, 50-200 mg TID, 300-600 mg TID, 200-400 mg TID, 200-600 mg TID, 100 to 700 mg twice daily (BID), 100-600 mg BID, 200-500 mg BID, or 200-300 mg BID. The amount of compound administered will depend on the potency of the compound of the present invention and the magnitude or effect desired and the route of administration.

A purified compound of the present invention (again, preferably provided as part of a pharmaceutical preparation) can be administered alone or in combination with one or more other therapeutic agents, such as other drugs for treating T cell exhaustion (e.g., tyrosine kinase inhibitors (e.g., dasatinib, ponatinib) (e.g., anti-PD-1 checkpoint inhibitor, such as nivolumab), or other medications used to treat a subject for an infection or disease associated with T cell exhaustion (e.g., antiviral, antibiotic, or anti-cancer drugs); or adoptive T cell therapies (e.g., a CAR T-cell therapy, a transduced T-cell therapy, and a tumor infiltrating lymphocyte (TIL) therapy); or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

A compound of the present invention can be administered prior to, concurrent with, or subsequent to other agents or therapies. If provided at the same time as other agents or therapies, one or more compounds of the present invention can be provided in the same or in a different composition. Thus, one or more compounds of the present invention and other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising a compound of the present invention and a dose of a pharmaceutical composition comprising at least one other agent, such as another drug for treating T cell exhaustion, which in combination comprise a therapeutically effective dose, according to a particular dosing regimen. Similarly, one or more compounds of the present invention and one or more other therapeutic agents can be administered in at least one therapeutic dose. Administration of the separate pharmaceutical compositions or therapies can be performed simultaneously or at different times (i.e., sequentially, in either order, on the same day, or on different days), as long as the therapeutic effect of the combination of these substances is caused in the subject undergoing therapy.

The invention also provides kits comprising one or more containers holding compositions comprising at least one compound of the present invention and optionally one or more other agents for treating T cell exhaustion. Compositions can be in liquid form or can be lyophilized. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions for methods of using the compositions comprising at least one compound of the present invention for treating a subject for T cell exhaustion. The package insert can be an unapproved draft package insert or can be a package insert approved by the Food and Drug Administration (FDA) or other regulatory body.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example I

This example describes CAR-T Tandem Mass Tag (TMT) proteomics.

In order to characterize the effect of the compounds of the present invention (e.g., compounds having a thiazole, imidazolepyridiazine or piperazinyl-methyl-aniline structure), detailed proteomics analysis of healthy donor purified T cells (e.g., from three human subjects) that were artificially conditioned to become exhausted ex vivo by transducing them to express a CAR that tonically signals in the absence of antigen was performed. The CAR-T cells were treated with Dastanib and N-(2-chloro-6-methylphenyl)-2-(4-methyl-3-(4-(pyridin-3-yl)pyrimidin-2-ylamino)benzamido)thiazole-5-carboxamide

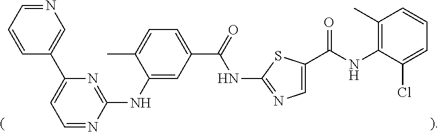 [SMILES: Cc1ccc(C(=O)Nc2ncc(C(=O)Nc3c(C)cccc3Cl)s2)cc1Nc1nccc(-c2cccnc2)n1]

From a collective analysis of over 1200 proteins (e.g., a Maxquant search was performed against the human Swiss-Prot database (Aug. 3, 2017, 42,210 entries)) the experiments resulted in identification of three proteins that increased and four that are decreased by treatment (see, Tables 1 and 2). Such results thereby yielded potential protein targets where the action of the compounds of the present invention leads to modulation of CAR Ts.

The following three proteins showing increased expression following exposure to Dastanib and N-(2-chloro-6-methylphenyl)-2-(4-methyl-3-(4-(pyridin-3-yl)pyrimidin-2-ylamino)benzamido)thiazole-5-carboxamide

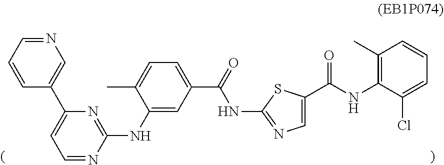 [SMILES: Cc1ccc(C(=O)Nc2ncc(C(=O)Nc3c(C)cccc3Cl)s2)cc1Nc1nccc(-c2cccnc2)n1]

were identified as optimal targets:
POLDIP2—DNA Polymerase Delta Interacting Protein 2, silencing increases sensitivity of cells to oxidative stress, regulates cell/mitochondrial metabolism;
GSTK1—Glutathione S-Transferase Kappa, cellular detoxification by removal of hydrophobic substances; and
STMN2—Stathmin 2 or Neuron-Specific Growth-Associated Protein, regulates microtubule dynamics and stability.

The following four proteins showing decreased expression following exposure to Dastanib and EB1P074 were identified as optimal targets:
GZMB—Granzyme B or T-Cell Serine Protease 1-3E, secreted by natural killer (NK) cells and cytotoxic T lymphocytes (CTLs) to induce target cell apoptosis;
MAPRE2—Microtubule Associated Protein RP/EB Family Member 2 or T-Cell Activation Protein, EB1 Family, spindle symmetry during mitosis, upregulated in activated T-cells;
NAMPT—Nicotinamide phosphoribosyltransferase or Pre-B Cell-Enhancing Factor, biosynthesis of NAD, NAMPT inhibitors kill T cells; and
SIGMAR1—Sigma 1-Type Opioid Receptor, modulates calcium signaling.

TABLE 1
Top 10 Protein Expression Increases Following Treatment with Dastanib and EB1P074

| 006_patient1 | 006_patient2 | 006_patient3 | DAS_patient1 | DAS_patient2 | DAS_patient3 |
|---|---|---|---|---|---|
| OAT | STMN2 | SLC9A3R1 | HLA-DRB1 | POLDIP2 | UBR4 |
| GSTK1 | STOML2 | YBX3 | GSTK1 | LYPLA2 | SMU1 |
| SAR1B | TMSB4X | ANXA7 | HIST1H2BN | FLII | PSPC1 |
| POLDIP2 | FLII | PSMB10 | HNRNPUL1 | S100A10 | MYO18A |
| HLA-DRB1 | POLDIP2 | UBR4 | NUCB1 | STMN2 | GSTK1 |
| CSK | S100A4 | ARHGDIB | MYBBP1A | TMSB4X | SLC9A3R1 |
| SF3B4 | TTLL12 | GDI1 | ETHE1 | ACO2 | STMN2 |
| LTA4H | S100A10 | MYL1 | OAT | CAPNS1 | STK25 |
| MYBBP1A | CAPNS1 | LIG1 | EIF4A3 | S100A4 | STAT1 |
| NSDHL | FAF2 | BSG | SAR1A | ADRBK1 | PSMD12 |

TABLE 2
Top 10 Protein Expression Decreases Following Treatment with Dastanib and EB1P074

| 006_patient1 | 006_patient2 | 006_patient3 | DAS_patient1 | DAS_patient2 | DAS_patient3 |
|---|---|---|---|---|---|
| GZMB | PAFAH1B3 | BCAP31 | GZMB | GZMB | BCAP31 |
| SNRPB2 | OAT | HIST1H2BN | ECHS1 | WDR12 | GZMB |
| SCOC | GZMB | GZMB | HNRNPU | MAPRE2 | NAMPT |
| DPP3 | SIGMAR1 | RAP1GDS1 | SCOC | SATB1 | MKI67 |
| HNRNPU | EED | LONP1 | NAMPT | UBA2 | MAPRE2 |
| NAMPT | PGM1 | CUTA | ADRBK1 | NAMPT | GFPT1 |
| SIGMAR1 | TM9SF2 | NAMPT | CLINT1 | SIGMAR1 | PEA15 |
| CD2 | NAMPT | ACTC1 | RBM25 | SLC27A2 | EIF1 |
| ALOX5AP | MAPRE2 | G3BP2 | MTPN | EED | QRICH1 |
| LUC7L2 | SRPR | CCT6B | RER1 | HEATR1 | EIF4A2 |

LC-MS/MS analysis. Each TMT six-plex sample was analyzed in triplicate on a LTQ-Orbitrap Elite mass spectrometer (Thermo Fisher Scientific) with a Dionex Ultimate 3000 LC (Thermo Fisher Scientific). Three microliters of sample were injected onto a 5 mm C18 PepMap100 column (ID: 300 μm, particle size: 5 μm, pore size: 100 Å, Thermo Fisher Scientific) for desalting prior to a PicoFrit self-pack analytical column (OD: 360 μm, ID: 75 μm, Tip: 15±1 μm, no coating, New Objective, Woburn, Mass.) packed with 25 cm of MagicC18 AQ (particle size: 5 μm, pore size: 100 Å, C18 resin, Michrom, Auburn, Calif.). Mobile phase A was 0.1% formic acid in water, and mobile phase B was 0.1% formic acid in acetonitrile. A flow rate of 0.6 μL/min was used for peptide separation over 100 minutes using a gradient of 2-35% B, followed by two minutes with a gradient of 35-85% B, and seven minutes at 85% B. MS1 data was acquired between 400-1800 m z in the Orbitrap with a resolution of 30,000, an AGC setting of 1e6, and a maximum inject time of 100 ms. Ions were selected for fragmentation using a top-eight, data-dependent method with a charge state requirement of 2+ or higher, a 4 m/z isolation window, and a dynamic exclusion window of 30 s. High energy collision-induced dissociation (HCD) was performed on these isolated precursors with a normalized collision energy of 35, 0.1 ms activation time, 100 ms maximum inject time, and an AGC setting of 5e4. MS2 data was acquired over a mass range of 110-2000 m/z in the Orbitrap with a resolution of 30,000.

Protein identification and quantitation. Peptide identification and quantitation was performed using MaxQuant version 1.6.0.1 and Perseus version 1.6.0.7 (Cox Lab, Max Planck Institute). Triplicate runs were analyzed together as fractions in MaxQuant against the human Swiss-Prot database (Aug. 3, 2017, 42,210 entries). The reporter ion MS2 method for TMT six-plex samples was used with a reporter ion mass tolerance of 0.003 Da. Specific digestion was selected with trypsin/P as the enzyme and a maximum of two missed cleavages allowed. The precursor and fragment mass tolerance was 20 ppm, and the minimum peptide length was five amino acids. Allowed variable modifications were oxidation at methionine, acetylation at the protein N-terminus, and glutamine or glutamic acid conversion to pyro-glutamic acid, with a maximum of five modifications allowed per peptide, and the only fixed modification was carbamidomethylation at cysteine residues. A 1% FDR for peptide and protein IDs was used from a target-decoy search using reverse peptide sequences. The razor protein ID was used in cases where multiple protein IDs could be made.

Results were filtered to remove contaminants (as identified by MaxQuant) and reverse sequence IDs. Fold changes were calculated as the ratios of corrected reporter ion intensities compared to control. The base two logarithm of these fold changes was calculated, and median centering was performed in Perseus.

### Example II

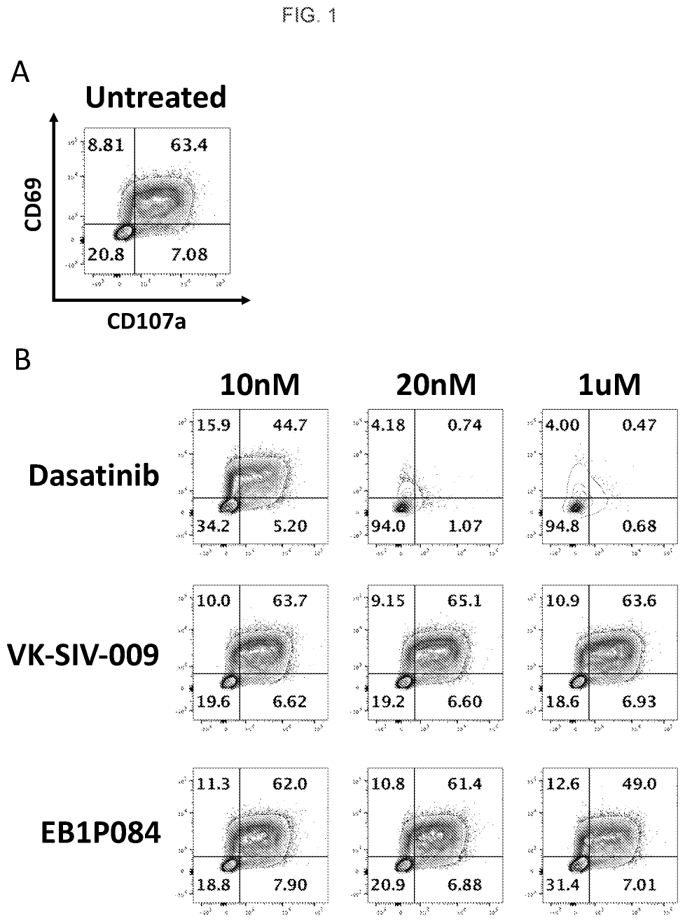

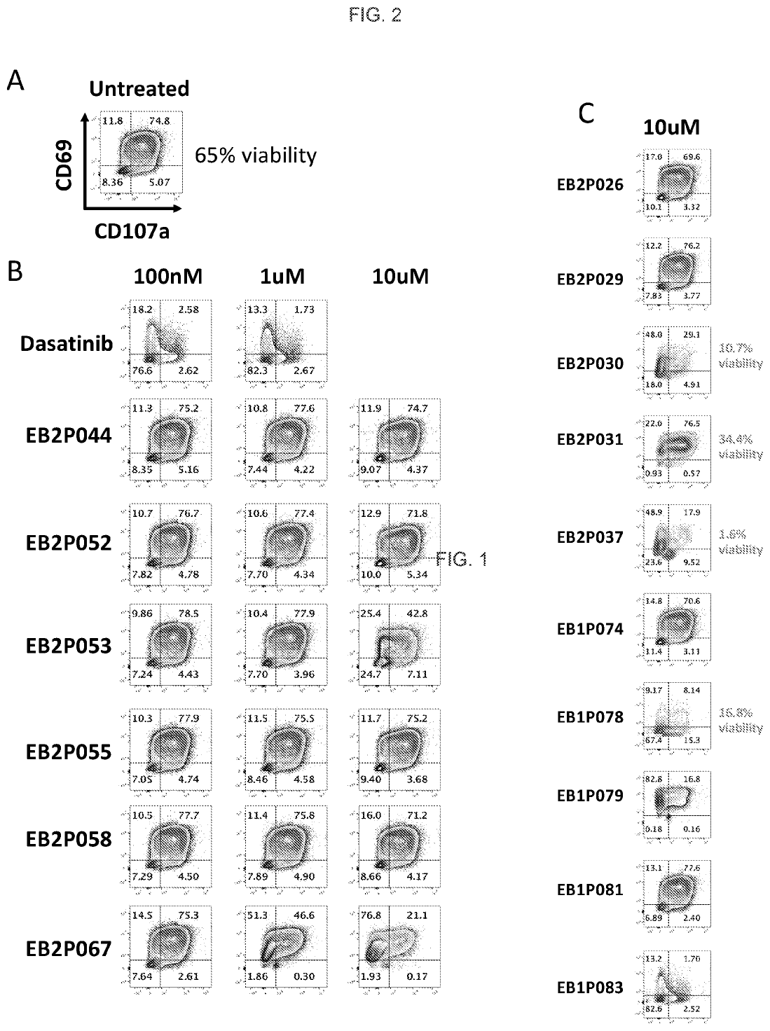

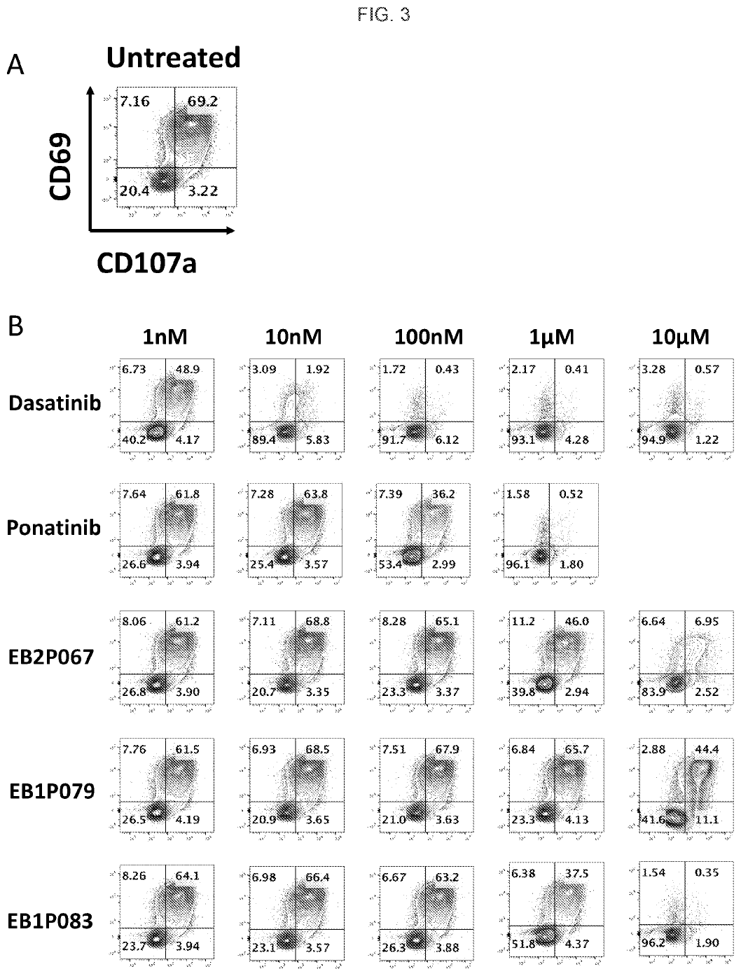

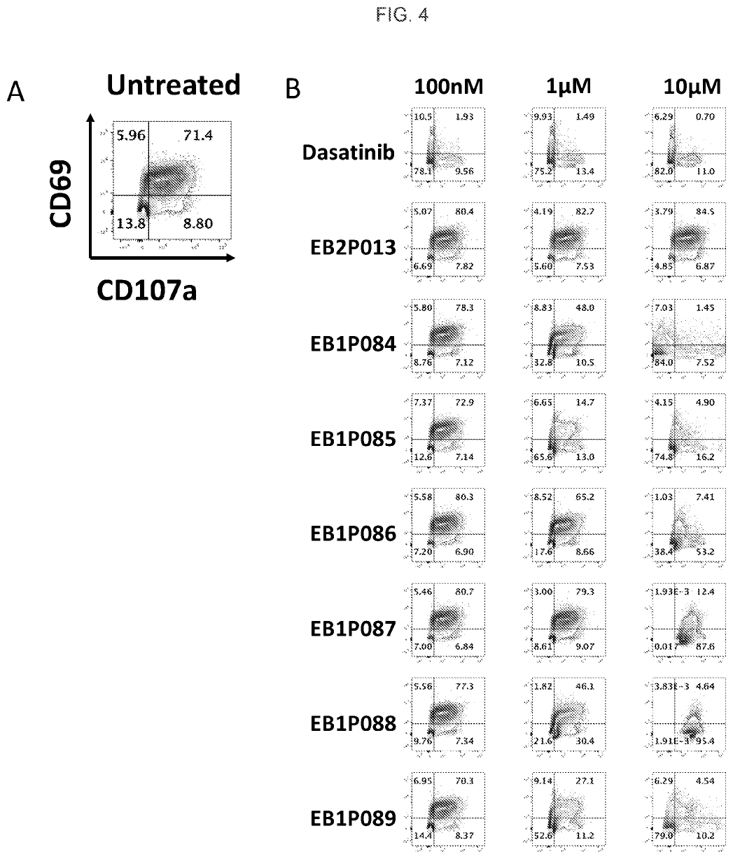

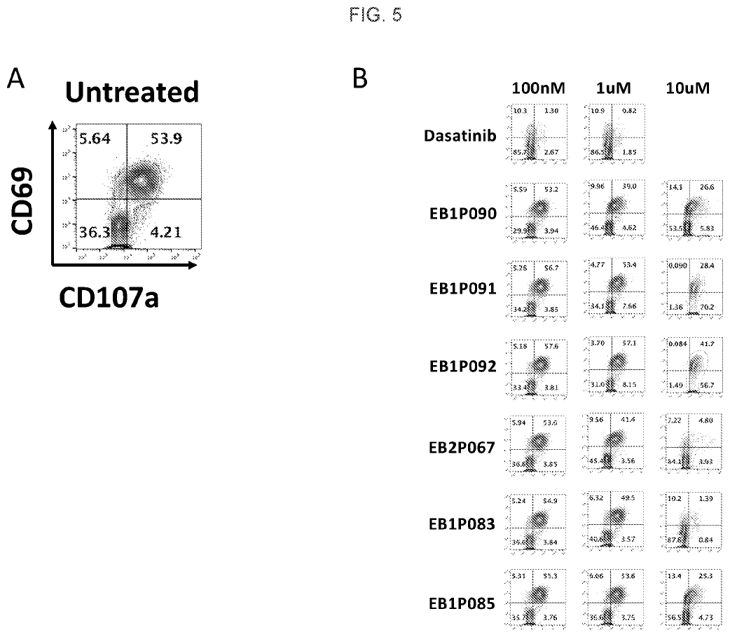

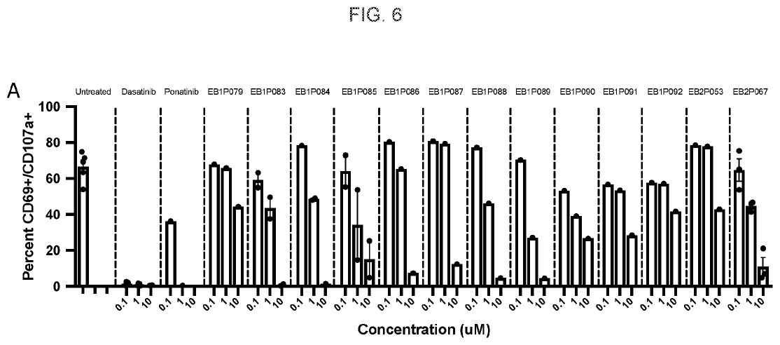

To assess the effects of novel compounds on CAR T cell antigen-induced activation, CD19.28ζ CAR-T cells were co-cultured with CD19-bearing Nalm6 leukemia cells that were engineered to express GFP and luciferase (Nalm6-GL) for 6 hours in the presence or absence of compounds, then used flow cytometry to measure surface expression of CD69, an early T cell activation marker, and CD107a, a surrogate marker for T cell degranulation. Dasatinib, which has been shown to potently inhibit CAR-T cell activation and anti-tumor function (see, Weber et al., Blood Adv, 2019), was used as a positive control for suppression of CD69/CD107a. Five independent experiments were conducted, wherein different combinations of novel compounds were tested at various dose-titrated concentrations (FIGS. 1-5), and a summary of these experiments is shown in FIG. 6.

Of the 27 novel compounds tested, 13 induced measurable suppression of CD69 and CD107a at the highest tested concentration of 10 uM, and 8 (EB1P083, EB1P084, EB1P085, EB1P086, EB1P088, EB1P089, EB1P090, EB1P091, EB2P067) induced measurable suppression at 1 uM. Of those, EB1P084, EB1P085, EB1P088, EB1P089, and EB2P067 exhibited the greatest potency at the 1 uM concentration compared to others.

### Production of Human CAR-T Cells

Primary human T cells were isolated using the RosetteSep Human T cell Enrichment kit (Stem Cell Technologies) and cryopreserved. T cells were thawed and activated with Human T-Expander CD3/CD28 Dynabeads (Gibco) at 3:1 bead:cell ratio in complete medium (AIMV supplemented with 5% FBS, 10 mM HEPES, 2 mM GlutaMAX, 100 U/mL penicillin (Gibco), and 100 U/mL (Peprotech)). T cells were transduced with retroviral vector on days 2 and 3 post-activation and maintained at 0.5-1×10⁶ cells/mL as previously described (see, Long et al., Nat. Med., 2015).

### Intracellular Cytokine Staining

CD19.28ζ CAR-T cells were cultured with dasatinib or new compounds for 24 hr prior to and for the duration of co-culture with Nalm6 cells stably expressing GFP and luciferase (Nalm6-GL). T cells were co-cultured with Nalm-6GL for 6 hours at a 1:1 effector:target ratio with Nalm6-GL in the presence of 1× monensin (eBioscience) and 1 uL/test CD107a antibody (BV605, Clone H4A3, BioLegend). Cells were washed and stained for CAR (anti-FMC63 idiotype antibody), live/dead, and anti-CD69(BV421 or PE, Clone FN50, Biolegend) for 30 minutes at 4 C. Cells were washed and prepared for analysis on a BD Fortessa cytometery running FACSDiva software.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound having Formula I, II or III: 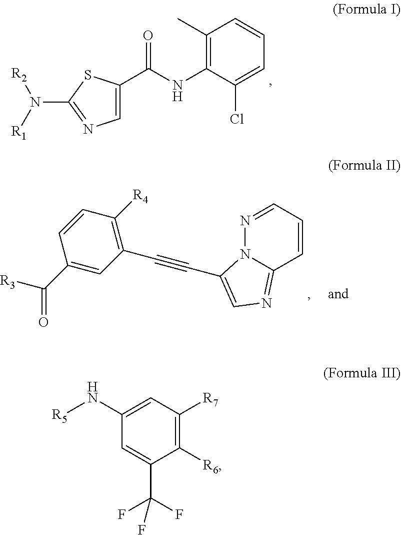
including pharmaceutically acceptable salts, solvates, and/or prodrugs thereof; wherein R1, R2, R3, R4, R5, R6 and R7 independently include any chemical moiety that renders the resulting compound capable of one or more of:
increasing CAR-T cell expression of POLDIP2;
increasing CAR-T cell expression of GSTK1;
increasing CAR-T cell expression of STMN2;
decreasing CAR-T cell expression of GZMB;
decreasing CAR-T cell expression of MAPRE2;
decreasing CAR-T cell expression of NAMPT;
decreasing CAR-T cell expression of SIGMAR1;
modulating (e.g., inhibiting) TCR or CAR-mediated signaling related to antigen-dependent or antigen-independent CAR T cell activation;
preventing and/or reversing T cell exhaustion related to antigen-dependent or antigen-independent CAR T cell activation;
decreasing CAR-T cell expression of one or more of PD-1, TIM-3, and LAG-3;
increasing CAR-T cell expression of memory markers (e.g., CD62L);
preventing CAR-T cell apoptosis;
decreasing CAR-T cell secretion of IL-2 and other cytokines; and
increasing CAR-T cell secretion of IL-2 and other cytokines after transient compound treatment and subsequent compound clearance.

2. The compound of claim 1,
wherein R1 and R2 are independently selected from hydrogen 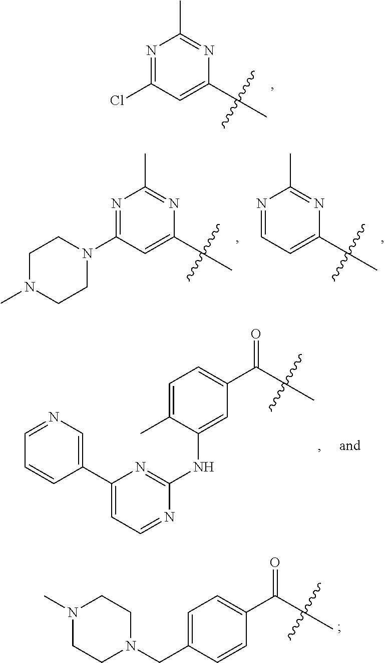
wherein R3 is selected from hydrogen, hydroxyl 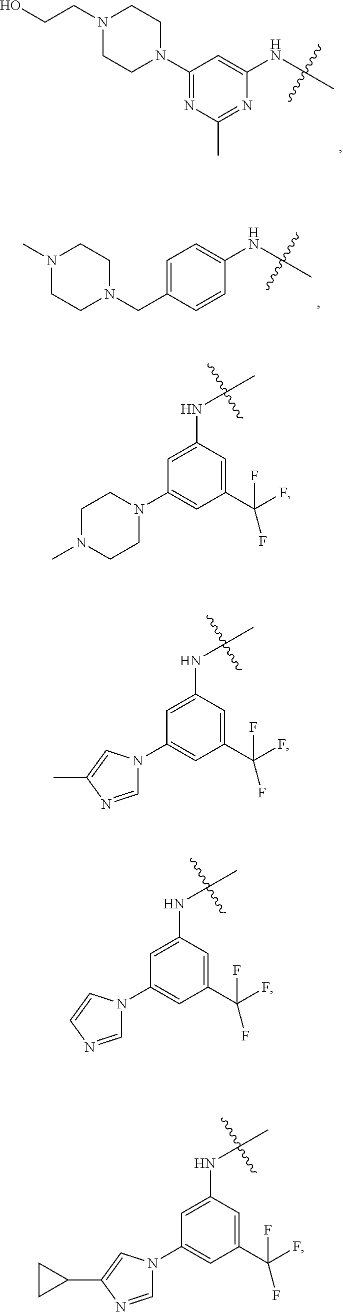 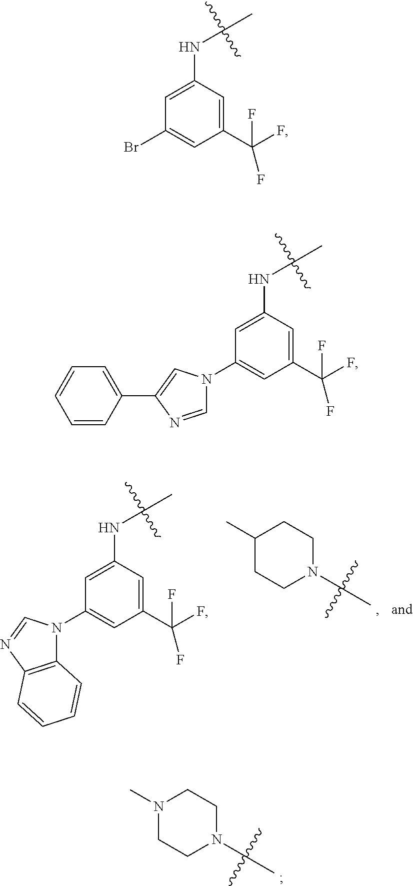
wherein R4 is hydrogen, methyl or 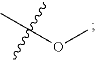
wherein R5 is selected from hydrogen 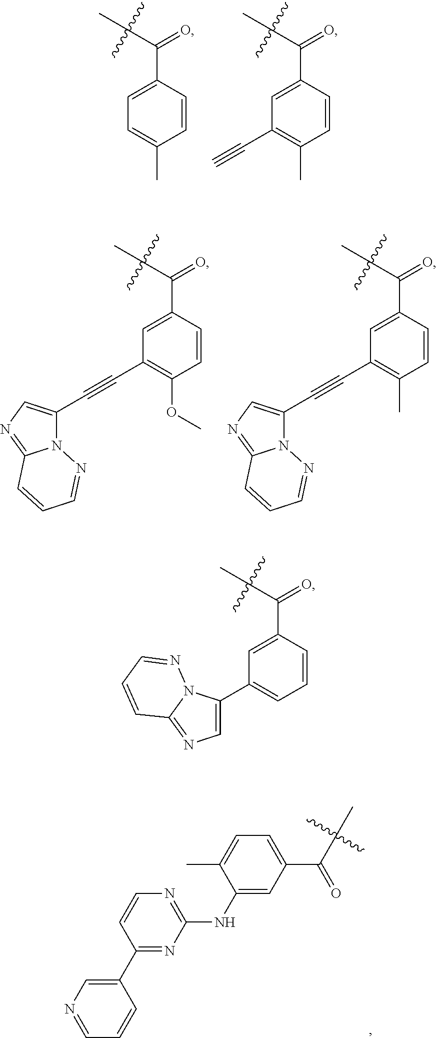 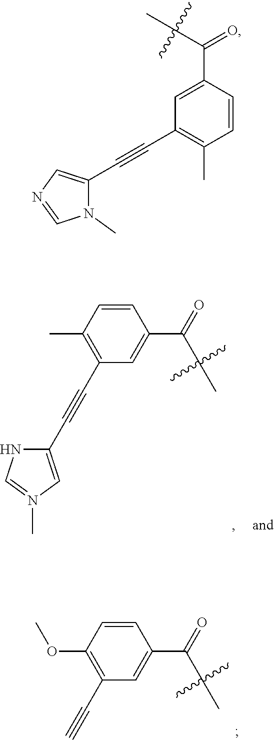
wherein R6 is hydrogen or 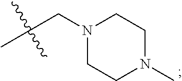
wherein R7 is hydrogen or

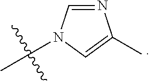 [SMILES: *n1cnc(C)c1]

3-7. (canceled)

8. The compound of claim 1, wherein said compound is selected from the group consisting of 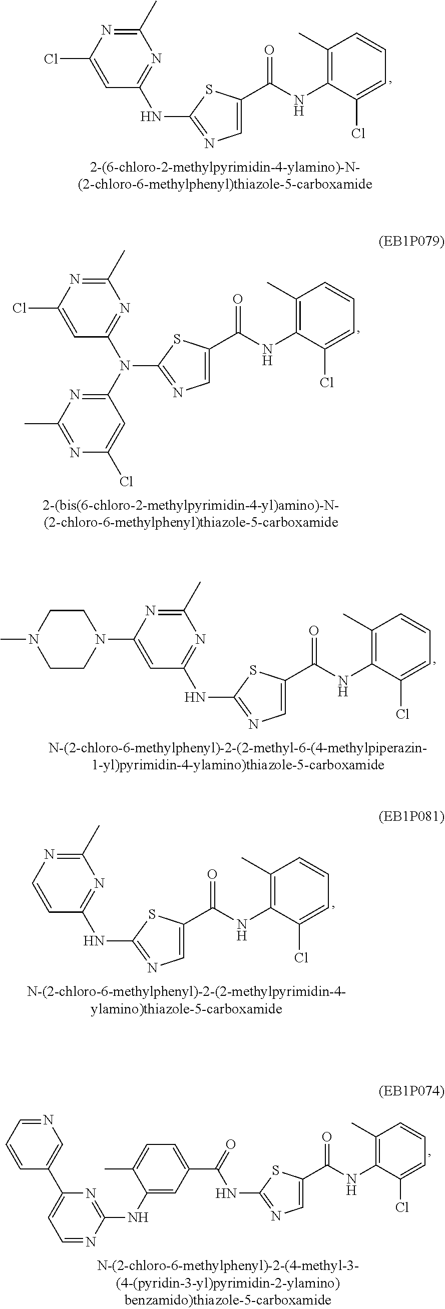 [SMILES: Cc1nc(Cl)cc(Nc2ncc(C(=O)Nc3c(C)cccc3Cl)s2)n1 ; Cc1nc(Cl)cc(N(c2cc(Cl)nc(C)n2)c2ncc(C(=O)Nc3c(C)cccc3Cl)s2)n1 ; Cc1nc(Nc2ncc(C(=O)Nc3c(C)cccc3Cl)s2)cc(N2CCN(C)CC2)n1 ; Cc1nccc(Nc2ncc(C(=O)Nc3c(C)cccc3Cl)s2)n1 ; Cc1ccc(C(=O)Nc2ncc(C(=O)Nc3c(C)cccc3Cl)s2)cc1Nc1nccc(-c2cccnc2)n1] 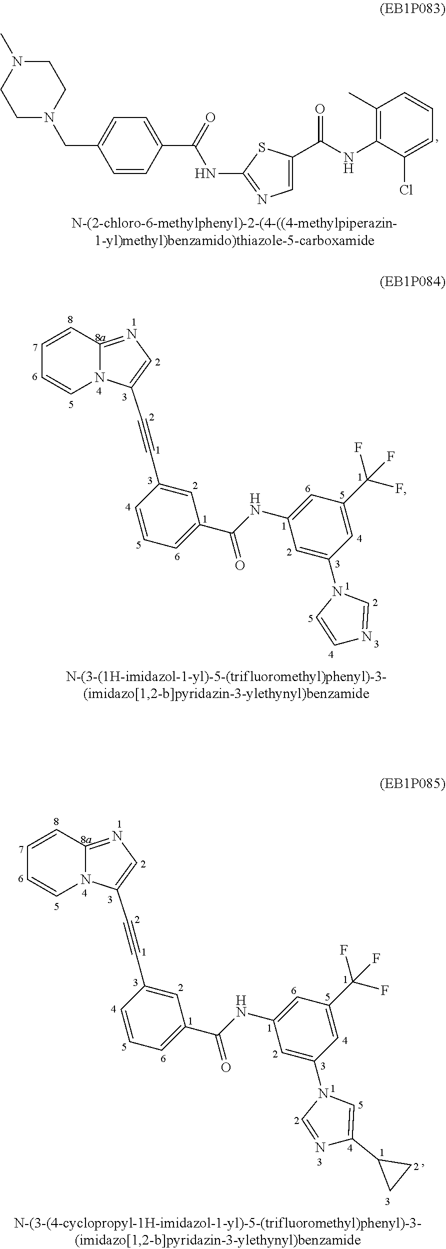 [SMILES: Cc1cccc(Cl)c1NC(=O)c1cnc(NC(=O)c2ccc(CN3CCN(C)CC3)cc2)s1 ; O=C(Nc1cc(-n2ccnc2)cc(C(F)(F)F)c1)c1cccc(C#Cc2cnc3ccccn23)c1 ; O=C(Nc1cc(-n2cnc(C3CC3)c2)cc(C(F)(F)F)c1)c1cccc(C#Cc2cnc3ccccn23)c1] 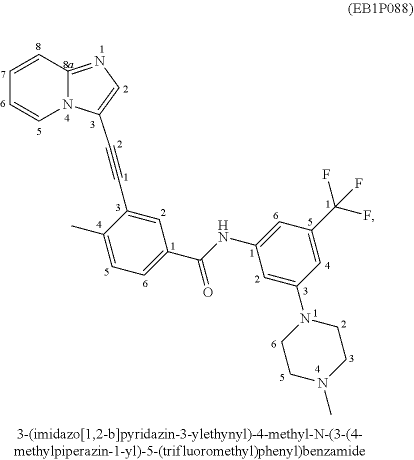 [SMILES: Cc1ccc(C(=O)Nc2cc(N3CCN(C)CC3)cc(C(F)(F)F)c2)cc1C#Cc1cnc2ccccn12] 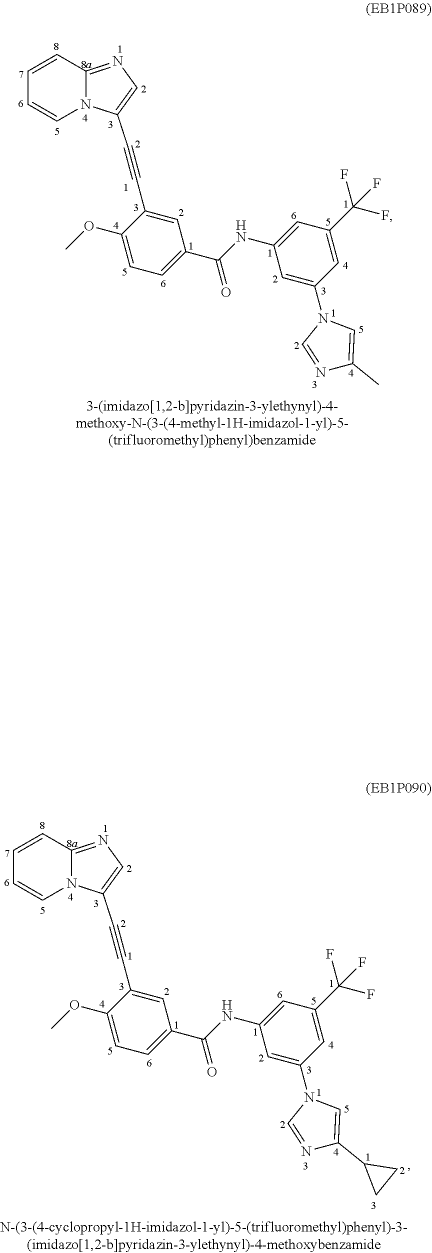 [SMILES: COc1ccc(C(=O)Nc2cc(-n3cnc(C)c3)cc(C(F)(F)F)c2)cc1C#Cc1cnc2ccccn12 ; COc1ccc(C(=O)Nc2cc(-n3cnc(C4CC4)c3)cc(C(F)(F)F)c2)cc1C#Cc1cnc2ccccn12] 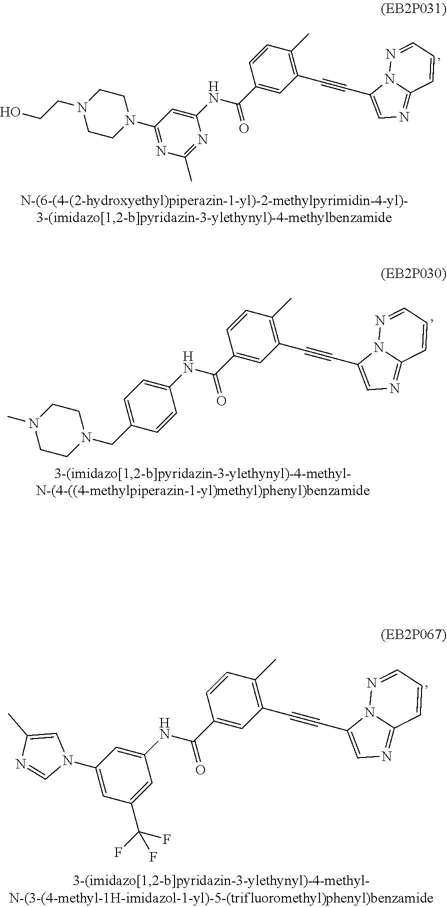 [SMILES: Cc1nc(NC(=O)c2ccc(C)c(C#Cc3cnc4cccnn34)c2)cc(N2CCN(CCO)CC2)n1 ; Cc1ccc(C(=O)Nc2ccc(CN3CCN(C)CC3)cc2)cc1C#Cc1cnc2cccnn12 ; Cc1cn(-c2cc(NC(=O)c3ccc(C)c(C#Cc4cnc5cccnn45)c3)cc(C(F)(F)F)c2)cn1] 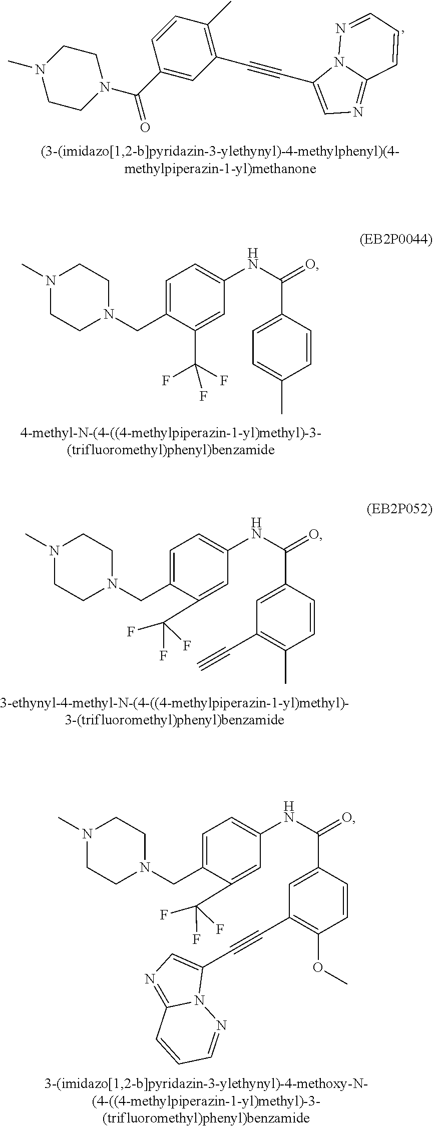 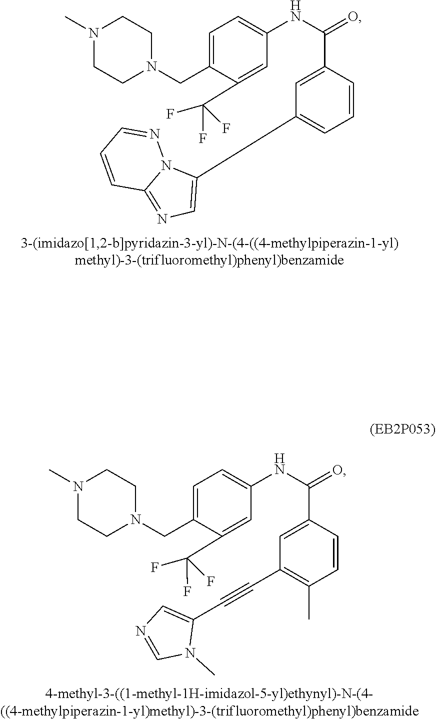 [SMILES: CN1CCN(Cc2ccc(NC(=O)c3cccc(-c4cnc5cccnn45)c3)cc2C(F)(F)F)CC1 ; Cc1ccc(C(=O)Nc2ccc(CN3CCN(C)CC3)c(C(F)(F)F)c2)cc1C#Cc1cncn1C] 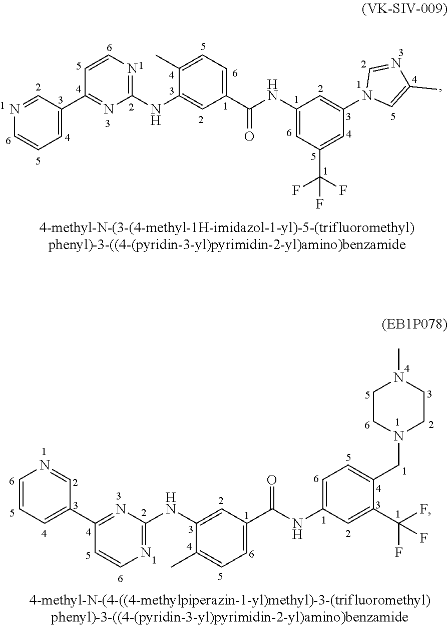 [SMILES: Cc1cn(-c2cc(NC(=O)c3ccc(C)c(Nc4nccc(-c5cccnc5)n4)c3)cc(C(F)(F)F)c2)cn1 ; Cc1ccc(C(=O)Nc2ccc(CN3CCN(C)CC3)c(C(F)(F)F)c2)cc1Nc1nccc(-c2cccnc2)n1] 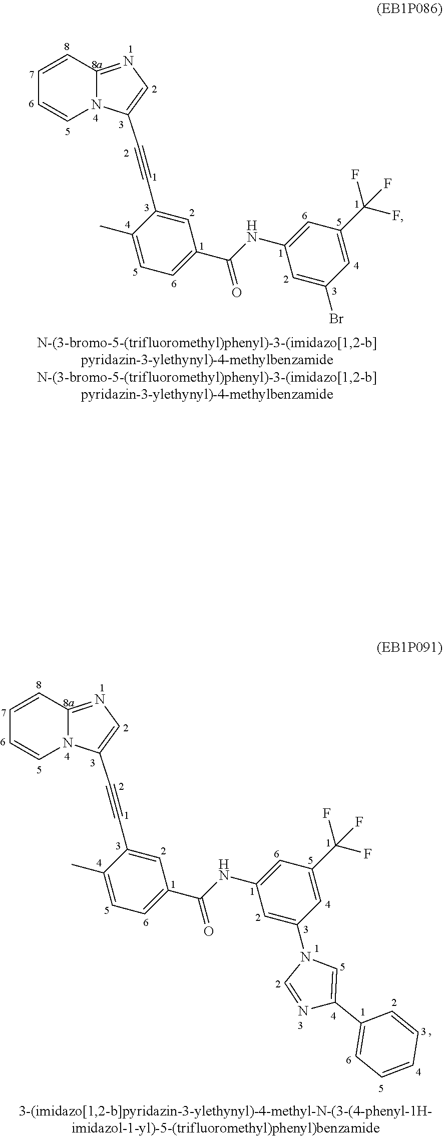 [SMILES: Cc1ccc(C(=O)Nc2cc(Br)cc(C(F)(F)F)c2)cc1C#Cc1cnc2ccccn12 ; Cc1ccc(C(=O)Nc2cc(-n3cnc(-c4ccccc4)c3)cc(C(F)(F)F)c2)cc1C#Cc1cnc2ccccn12] 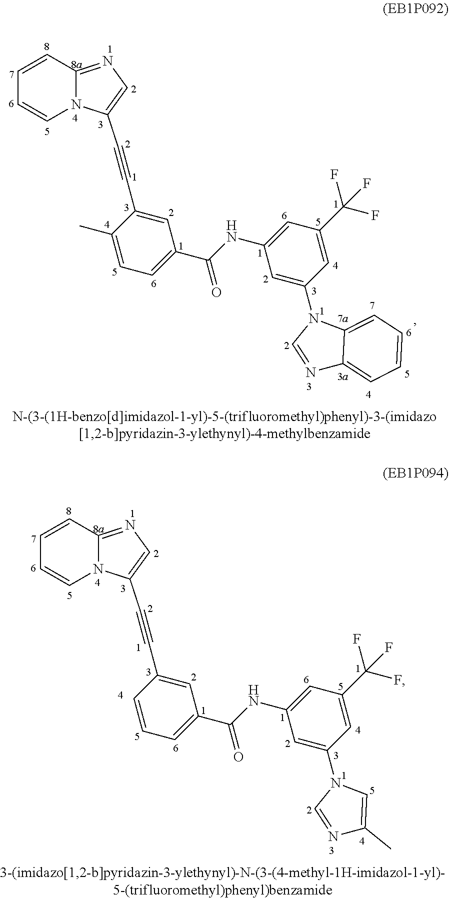 [SMILES: Cc1ccc(C(=O)Nc2cc(-n3cnc4ccccc43)cc(C(F)(F)F)c2)cc1C#Cc1cnc2ccccn12 ; Cc1cn(-c2cc(NC(=O)c3cccc(C#Cc4cnc5ccccn45)c3)cc(C(F)(F)F)c2)cn1] 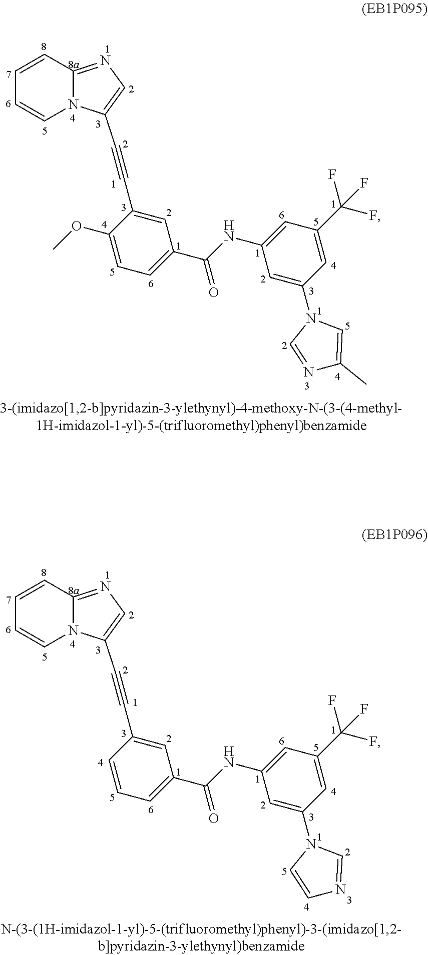 [SMILES: COc1ccc(C(=O)Nc2cc(-n3cnc(C)c3)cc(C(F)(F)F)c2)cc1C#Cc1cnc2ccccn12 ; O=C(Nc1cc(-n2ccnc2)cc(C(F)(F)F)c1)c1cccc(C#Cc2cnc3ccccn23)c1] 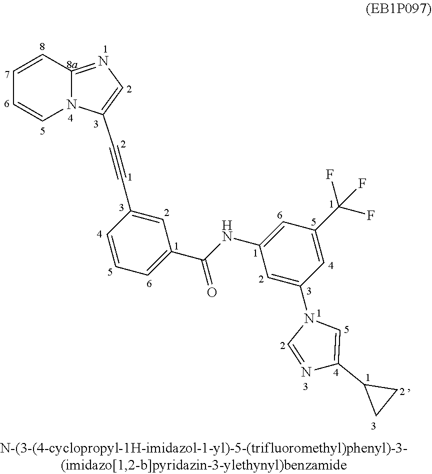 [SMILES: O=C(Nc1cc(-n2cnc(C3CC3)c2)cc(C(F)(F)F)c1)c1cccc(C#Cc2cnc3ccccn23)c1] 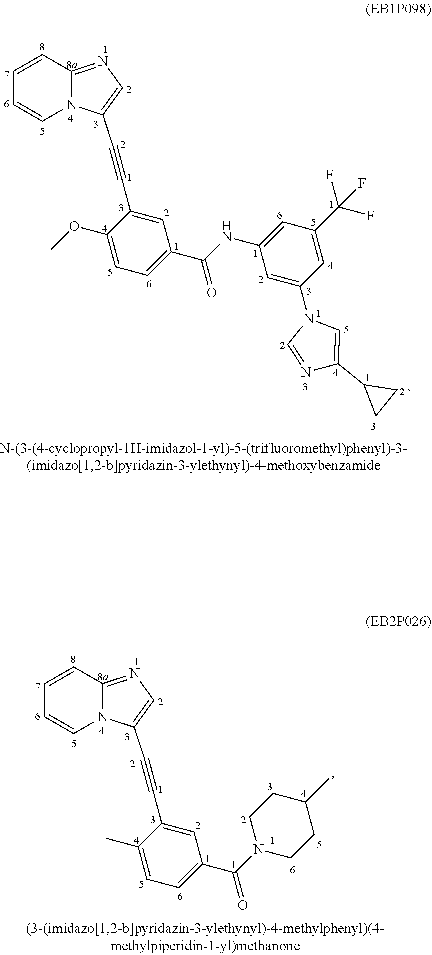 [SMILES: COc1ccc(C(=O)Nc2cc(-n3cnc(C4CC4)c3)cc(C(F)(F)F)c2)cc1C#Cc1cnc2ccccn12 ; Cc1ccc(C(=O)N2CCC(C)CC2)cc1C#Cc1cnc2ccccn12] 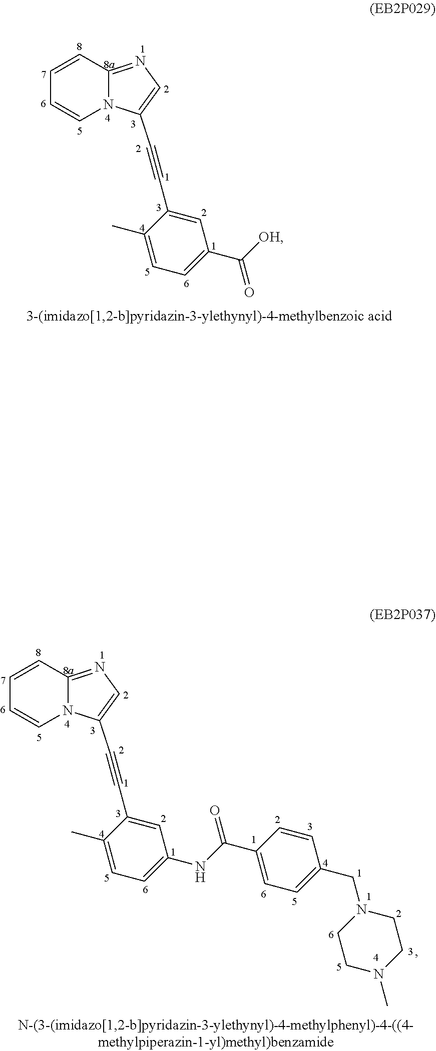 [SMILES: Cc1ccc(C(=O)O)cc1C#Cc1cnc2ccccn12 ; Cc1ccc(NC(=O)c2ccc(CN3CCN(C)CC3)cc2)cc1C#Cc1cnc2ccccn12] 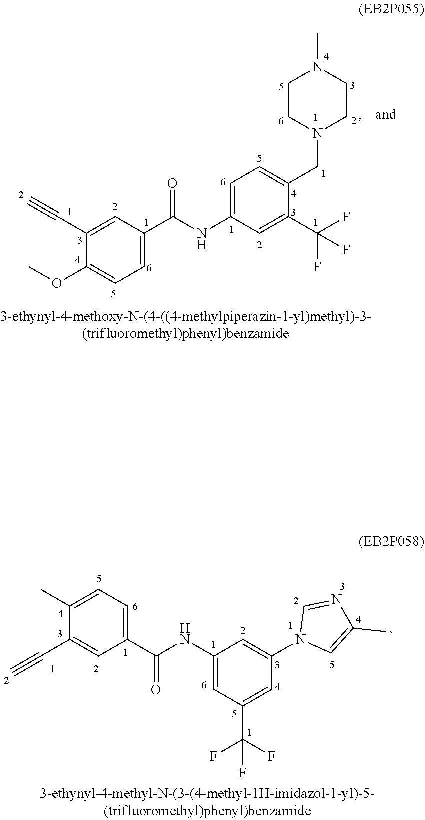 [SMILES: C#Cc1cc(C(=O)Nc2ccc(CN3CCN(C)CC3)c(C(F)(F)F)c2)ccc1OC ; C#Cc1cc(C(=O)Nc2cc(-n3cnc(C)c3)cc(C(F)(F)F)c2)ccc1C]
including pharmaceutically acceptable salts, solvates, and/or prodrugs thereof.

9-22. (canceled)

23. A method for treating an immune system related condition or disease in a subject comprising administering to the subject genetically engineered T cells and a therapeutically effective amount of a pharmaceutical composition comprising a compound of claim 1.

24. The method of claim 23, wherein the pharmaceutical composition and the genetically engineered T cells are administered simultaneously and/or at different time points.

25. The method of claim 23, wherein the immune system related condition or disease is selected from cancer or an autoimmune disease or condition.

26. The method of claim 23, wherein the genetically engineered T cells are selected from CAR T cells, genetically engineered TCR expressing T cells, genetically engineered T cells configured for tumor infiltrating lymphocyte (TIL) therapy, genetically engineered T cells configured for transduced T-cell therapy, and/or viral specific T cells reengineered with a TCR or CAR.

27. The method of claim 23, further comprising administering to said subject one or more anticancer agents.

28. The method of claim 27, wherein the one or more anticancer agents is selected from a chemotherapeutic agent and radiation therapy.

29. The method of claim 23, further comprising administering to said subject a tyrosine kinase inhibitor.

30. The method of claim 29, wherein the tyrosine kinase inhibitor is capable of inhibiting TCR signaling and/or CAR signaling.

31. (canceled)

32. The method of claim 29, wherein the tyrosine kinase inhibitor is a Lck inhibitor.

33. The method of claim 29, wherein the tyrosine kinase inhibitor is dasatinib or ponatinib.

34. The method of claim 23, wherein the pharmaceutical composition is administered orally.

35. The method of claim 23, wherein the subject is human.

36. A composition comprising a genetically engineered T cell population, wherein the genetically engineered T cell population was expanded in the presence of a compound of claim 1.

37. The composition of claim 36, wherein the genetically engineered T cell population was further expanded in the presence of a tyrosine kinase inhibitor capable of inhibiting TCR signaling and/or CAR signaling.

38. The composition of claim 37, wherein the tyrosine kinase inhibitor is a Lck inhibitor.

39. The composition of claim 37, wherein the tyrosine kinase inhibitor is dasatinib or ponatinib.

40. The composition of claim 36, wherein the genetically engineered T cell population is selected from CAR T cell population, a population of genetically engineered TCR expressing T cells, a population of genetically engineered T cells configured for tumor infiltrating lymphocyte (TIL) therapy, a population of genetically engineered T cells configured for transduced T-cell therapy, and/or a population of viral specific T cells reengineered with a TCR or CAR.

41-69. (canceled)
